(19) United States
(12) Patent Application Publication
(10) Pub. No.: US 2026/0108683 A1
(43) Pub. Date: Apr. 23, 2026

(54) MEDICAMENT DISPENSER FOR HOLDING ELONGATE FORM MEDICAMENT CARRIER AND METHOD OF ASSEMBLING THEREOF

(71) Applicant: Merxin Limited, King's Lynn (GB)

(72) Inventors: Adam STUART, Pott Row (GB); Stephen HOWGILL, Leicestershire (GB)

(73) Assignee: Merxin Limited, King's Lynn, NF (GB)

(21) Appl. No.: 19/427,427

(22) Filed: Dec. 19, 2025

Related U.S. Application Data
(63) Continuation-in-part of application No. 18/713,267, now Pat. No. 12,539,375.

(30) Foreign Application Priority Data
Nov. 29, 2021 (GB) 2117176.4

(51) Int. Cl. A61M 15/00; A61J 1/03

(52) U.S. Cl. CPC A61M 15/0051; A61J 1/035; A61M 15/0001; A61M 15/0043; A61M 15/0061; A61M 2202/0007; A61M 2202/064; A61M 2205/8281

(57) ABSTRACT

Disclosed is a medicament dispenser for holding at least one elongate form medicament carrier. The elongate form medicament carrier has proximal and distal ends, and blister and lid elongate sheets enclosing multiple distinct medicament dose portions therebetween. The medicament dispenser has a dispensing mechanism which is adapted to operate, upon each actuation thereof, to dispense medicament active from the medicament dose portion. Moreover, the mechanism has at least one power spring mechanism for winding the lid elongate sheet, wherein the at least one power spring mechanism has at least one spring having an inner end about which the spring unwinds, and at least one shaft at which the inner end of at least one spring is secured. Furthermore, the power spring mechanism tensions and peels a segment of lid elongate sheet peeled from a corresponding segment of blister elongate sheet upon each actuation of the medicament dispenser. Disclosed also is a method of assembling the aforementioned medicament dispenser.

### TECHNICAL FIELD

The present invention relates to a medicament dispenser for releasing medicament doses. More specifically, the present invention relates to a medicament dispenser for holding elongate form medicament carriers.

### BACKGROUND

Conventionally, dry powder inhalation (DPI) devices are employed for dispensing powder form doses. One type of DPI device administers doses by means of elongate form carriers such as blister strips, which contain a number of discrete doses of medicament in powder form. Notably, this specific design of the elongate form carrier (i.e., an elongate lid sheet or film sealing a blister sheet comprising blisters having or containing medicaments) prevents the doses from contamination or degradation by moisture absorption. One sub-type of these devices releases a dose by puncturing the blister and scouring the contents with an airflow generated by the patient. In another sub-type, the lid sheet is peeled away from the blister sheet to expose a dose, again with the contents scoured with an airflow generated by the patient. In order to release the doses from the blisters of this sub-type of device, the lid sheet must be pulled taut and separated from the blister sheet. If the lid sheet is not held taut during peeling, it could become loose, or become out of synchronisation with the dispensing mechanism, resulting in a failed dosing. However, if the lid sheet is held too tightly it could stretch, become loose, break or release the dose too early. These factors may also contribute to an increase in the tendency of mechanical failure for such medicament dispensers. It is therefore critical to ensure the correct handling of lid sheet tensioning during inhaler actuation.

One way of controlling tension of the elongate sheets uses a blister sheet take-up spool and a lid sheet take-up capstan and flexible spring blades to maintain tension on the lid sheet. Operatively, the flexible spring arms are compressed as more lid sheet is spooled onto the capstan. Moreover, said compression is supposed to allow for a constant outer take-up capstan diameter throughout usage of the inhaler device. However, such a property relies on a balancing of the tensions in the unwound used sheet and the flexible spring arms while this sheet and the arms are increasingly separated by windings of used sheet. Furthermore, compression of a plastic component (the flexible spring arms) is maintained for an extended period. If the flexible spring arms do not compress enough, it may result in over-advancement of the lid sheet. Moreover, if the tension created by the capstan is too high the lid sheet might stretch, thus losing tension and cause the sheet to fracture, or pull too far during an actuation, thereby exposing the medicament for the next actuation too early as well as exposing the medicament to atmospheric conditions. Additionally, if the flexible spring arms weaken, some tension in the lid sheet will be lost and the mechanism may jam.

In another disclosure (EP1786498A1), a torsion spring of a sheet driver acts to compensate for any increase in the diameter of the effective winding surface of a fixed-diameter hub or bobbin during winding of sheet thereabout. Provided at a base of the sheet driver, there is a drive surface for receipt of drive to rotate the base about the rotational axis. A first leg receiver of the base receives a first spring leg of the torsion spring and a second leg receiver of the hub or bobbin receives a second spring leg of the torsion spring such that relative rotation of the base and the bobbin results in tensioning of the torsion spring. As lid sheet winds up around the bobbin the effective winding surface increases and the torsion spring acts such as to compensate for that increase.

In another device of the first sub-type, in which a blister is punctured, a spring is used in a receiver element for taking up used sheet. In such a fluid dispensing device the used elongate carrier is coiled onto a receiver element. However, such a device includes two blister sheet displacement mechanisms and requires the blister sheet having the medicament to be punctured at the site of dispensing to release the medicament, rather than the lid sheet being peeled away; so, the function of the receiver element is merely to take up the loose used elongate carrier.

Therefore, in light of the foregoing discussion, there exist opportunities to provide simpler mechanisms than are found in medicament dispensers of the prior art, and to provide an efficient, reliable, cost-effective and easy-to-use medicament dispenser.

### SUMMARY

The present disclosure seeks to provide a medicament dispenser for holding at least one elongate form medicament carrier. The present disclosure seeks to provide a solution to the existing problem of complex conventional medicament dispensers and potential failure associated therewith during progression of the medicament carrier in the medicament dispenser. An aim of the present disclosure is to provide a solution that overcomes at least partially the problems encountered in prior art, and provides a reliable, effective, robust mechanism for dispensing medicament doses while maintaining the integrity of the medicament carrier during progression thereof in the medicament dispenser.

The present disclosure is particularly applicable for inhalation delivery, or an inhalation delivery device, in particular a dry powder inhaler.

Embodiments of the present disclosure provide improvements over the prior art, and enable delivery of multiple distinct medicament doses in a single combined dosage whilst reducing rucking or over tensioning of the elongate form medicament carrier and misfeeding the elongate form medicament carrier around the receiving capstan. They also improve spooling of the peeled lid elongate sheet around the bobbin by way of the power spring mechanism, thereby better controlling the tension in the medicament carrier. Moreover, the disclosed medicament dispenser employs a common mechanism, i.e. peeling off the lid elongate sheet from the blister elongate sheet to dispense the medicament as well as effectively spooling the peeled lid elongate sheet around the bobbin by way of the power spring mechanism, and the peeled blister elongate sheet on the take-up spool. Furthermore, control of the tension in the lid elongate sheet by the power spring mechanism is independent of any gear mechanism to drive the receiving capstan, such that spooling of the lid elongate sheet cannot get out of step with spooling of the blister elongate sheet.

Additional aspects, advantages, features and objects of the present disclosure will be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIGS. 1A, 1B, 1C and 1D are schematic views of a power spring mechanism for use with a medicament dispenser, in accordance with an embodiment of the present disclosure;

FIG. 1E is a sectional schematic view of a power spring mechanism for use with a medicament dispenser, in accordance with an embodiment of the present disclosure;

FIG. 1F is a schematic view of a ratchet mechanism, suitable for incorporation into a medicament dispenser;

FIGS. 2A, 2B are schematic illustrations of the steps of anchoring and tensioning a distal end of the lid elongate sheet by or near to a circumference of a bobbin, in accordance with an embodiment of the present disclosure;

FIGS. 3A and 3B, are external views of opposite sides of a holder of a medicament dispenser, in accordance with an embodiment of the present disclosure;

FIG. 4 is a perspective view of an elongate form medicament carrier, in accordance with an embodiment of the present disclosure;

FIG. 5 is a sectional view of part of a medicament dispenser, in accordance with an embodiment of the present disclosure;

FIG. 6 is a perspective view of an elongate form medicament carrier, in accordance with another embodiment of the present disclosure; and

FIGS. 7 and 8 are sectional views of part of a medicament dispenser, in accordance with another embodiment of the present disclosure.

FIG. 9 shows a perspective view from one side and slightly above of two elongate form medicament carriers and associated bobbin units that form part of a medicament dispenser, the outer end of a lid elongate sheet that forms part of a medicament carrier shown heat-sealed to an associated bobbin that forms part of the bobbin unit.

FIG. 10 shows a perspective view from above of a bobbin unit and lid elongate sheet, the lid elongate sheet wound around a bobbin that forms part of the bobbin unit, the end of the lid elongate sheet taped to the bobbin using medical-grade tape.

FIG. 11 shows a perspective view from one side and slightly above of two elongate form medicament carriers and associated bobbin units that form part of a medicament dispenser, the outer end of a lid elongate sheet that forms part of a medicament carrier shown ultrasonically welded to an associated bobbin that forms part of the bobbin unit.

FIG. 12 shows a perspective view from one side and slightly above of two elongate form medicament carriers and associated bobbin units that form part of a medicament dispenser, the outer end of a lid elongate sheet that forms part of a medicament carrier shown glued to an associated bobbin that forms part of the bobbin unit.

FIGS. 13A and 13B show perspective views of bobbin units and connected lid elongate sheets, the bobbins of the bobbin units formed so as to comprise bobbin pegs around which the lid elongate sheets can be passed so as to wrap the ends of the lid elongate sheets around a bobbin peg, the lid elongate sheets then doubled back around the peg and secured to itself using a suitable securing method.

FIG. 14 shows a perspective view from one side and slightly above of a bobbin unit formed with an integral mechanical clip, the lid elongate sheet gripped in use by the clip to connect this to a bobbin that forms part of the bobbin unit.

FIG. 15A shows a perspective view from one side and slightly above of a bobbin unit configured with a plurality of short moulded or machined spikes or barbs protruding from the surface of a bobbin that forms part of the bobbin unit.

FIG. 15B shows a perspective view of the bobbin unit of FIG. 15A in use with a lid elongate sheet, the spikes or barbs piercing the lid elongate sheet and retaining it in position on the bobbin.

FIG. 16A shows a perspective view from one side and slightly above of a bobbin unit configured with a plurality of pegs protruding from the surface of a bobbin that forms part of the bobbin unit, at or towards the upper and lower sides of the bobbin.

FIG. 16B shows a perspective view of the bobbin unit of FIG. 16A in use with a lid elongate sheet, the lid elongate sheet pre-prepared with holes or apertures complementary to the pegs, the pegs protruding through the holes to retain the lid elongate sheet in position on the bobbin.

FIG. 16C shows a perspective view from one side and slightly above of a variation of the bobbin unit of FIGS. 16A and 16B, the pegs protruding from the surface of the bobbin at the centre of the bobbin.

FIG. 17a shows a perspective view from one side and slightly above of two elongate form medicament carriers and associated bobbin units that form part of a medicament dispenser, the outer end of a lid elongate sheet that forms part of a medicament carrier shown held in position on a bobbin that forms part of the bobbin unit, by way of a circlip.

FIG. 17b shows a perspective view from one side and above of a medicament carrier and a bobbin unit as shown in FIG. 17a, showing detail of the connection between the circlip and the bobbin.

FIG. 18 shows a perspective view of an elongate form medicament carrier that comprises a blister elongate sheet and a lid elongate sheet that enclose multiple distinct medicament dose portions therebetween, the end of the lid elongate sheet comprising a formed recess configured to receive a complementary feature on a bobbin so as to connect the elongate form medicament carrier to the bobbin in use.

In the accompanying drawings, a non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art will recognize that other embodiments for carrying out or practising the present disclosure are also possible.

The present disclosure provides the aforementioned medicament dispenser. The disclosed system ensures accurate peeling of a lid elongate sheet from a blister sheet, by a predictable mechanical mechanism. In this regard, the medicament dispenser comprises a spring that is not actively driven during use, and which moves naturally from a stressed state to a relatively unstressed state so as to maintain tension within a suitable range on the lid elongate sheet. Additionally, the spring enables the medicament dispenser to perform both the function of peeling the lid elongate sheet from the blister elongate sheet to expose (namely, dispense) medicament, as well as spooling the lid elongate sheet over a bobbin by use of the power spring mechanism, for a compact waste storage.

The medicament dispenser is a robust, user-friendly, and simple to manufacture and assemble system with an optimal performance thereof. The medicament dispenser effectively spools the lid elongate sheet and eliminates slack thereof throughout the usage of the medicament dispenser.

In an embodiment, beneficially, the medicament dispenser effectively exposes the medicament upon each actuation of the medicament dispenser, thereby, preventing the blister elongate sheet from exposing the medicament in advance and leaving it liable to become contaminated and/or degraded by atmospheric conditions thereafter. The receiving capstan is configured to hold medicament dose portions in fixed positions until actuation of the medicament dispenser.

In another embodiment, the medicament dispenser is designed to hold more than one (e.g. two or more) elongate form medicament carriers with relatively greater ease, thereby simplifying the assembly of the medicament dispenser and reducing the mechanisms required to drive the dispensing mechanism during use. Furthermore, such design of the medicament dispenser enables the dispensing of multiple bioactive components that may be taken in by the user in combination or sequentially. Accordingly, the medicament dispenser may comprise two or more power spring mechanisms, one for each elongate form medicament carrier.

Pursuant to embodiments of the present disclosure, there is provided a medicament dispenser for holding at least one elongate form medicament carrier. The term “elongate form medicament carrier” as used herein refers to a strip (or portion of a strip) that is fabricated from a thin sheet of flexible material or sheets of flexible materials, and which bears medicament (namely, medicament formulation or medicament product). Such medicament carriers may be used to administer a prescribed medicament (namely, medicament active or medicament actives) to an intended user, for example a patient suffering from a respiratory illness and associated symptoms. It will be appreciated that a plurality of such elongate form medicament carriers could be employed to administer a plurality of medicament actives, in combination or sequentially, useful for a variety of conditions such as asthma, pulmonary diseases such as COPD (chronic obstructive pulmonary disease), and so forth. The plurality, e.g., two, elongate form medicament carriers may be manufactured as a single strip.

The at least one elongate form medicament carrier has a proximal end, a distal end, and a blister elongate sheet and a lid elongate sheet enclosing multiple distinct medicament dose portions therebetween. The proximal end and the distal end are opposite to each other. It may be appreciated that the proximal end may refer to an end near to the bulk of the unused elongate form medicament carrier and the distal end may refer to an end that is farther away from the bulk of the unused elongate form medicament carrier. Moreover, the elongate form medicament carrier comprises a pair of elongate sheets (namely, the blister elongate sheet and the lid elongate sheet), that have been attached together, between the proximal end and the distal end. Furthermore, the blister elongate sheet has multiple distinct medicament dose portions for storing a medicament active, while the lid elongate sheet, corresponding to the size of the blister elongate sheet, covers or seals the multiple distinct medicament dose portions (carrying medicament actives therein) on the blister elongate sheet.

The term “medicament dose portion” as used herein refers to a measured quantity contained within a blister of the blister elongate sheet. The administrable dose is a whole number multiple of the medicament dose portion. Usually, one administrable dose can be provided in one medicament dose portion. However, it may be desirable to provide the administrable dose in two or more medicament dose portions. The administrable dose of the single medicament may be provided in at least one blister and combined with an administrable dose of another single medicament to provide a final dose for the patient. Alternatively, at least one of the administrable doses may comprise two or more medicaments.

Notably, the proximal end of the elongate form medicament carrier is employed to carry the multiple distinct medicament dose portions for carrying the medicament active. Optionally, the multiple distinct dose portions are uniformly spaced on the elongate form medicament carrier starting from the proximal end thereof. Optionally, the spacing of the multiple distinct medicament dose portions in two or more elongate form medicament carriers may be similar or different from each other. Notably, the distal end of the elongate form medicament carrier does not comprise multiple distinct medicament dose portions for carrying the medicament active and appears as a flat foil, optionally, with a looped end thereof.

Optionally, the elongate form medicament carrier may be manufactured from a plastic material, an aluminium foil, a combination thereof, or any other suitable material, and may comprise recesses for forming pockets or blisters. Optionally, the blister elongate sheet and the lid elongate sheet could be manufactured from a same or different fabrication material.

Optionally, the lid elongate sheet is fabricated from at least one of: a paper, a polyester, an aluminium foil. Optionally, the blister elongate sheet is fabricated from at least one of: an oriented polyamide (OPA), an aluminium foil, a polymeric material. Optionally, both the lid elongate sheet and the blister elongate sheet are fabricated as a multi-layered construction using a combination of metal, paper and polymeric materials. Beneficially, such multi-layered construction ensures a strong and reliable implementation of the lid elongate sheet and blister elongate sheet, thereby providing a superior barrier layer between the external contaminants and the medicament actives. Moreover, the lid elongate sheet is coated with a heat seal lacquer for bonding to the blister elongate sheet. Beneficially, the heat seal lacquer provides a sturdy hermetic sealing between the lid elongate sheet and the blister elongate sheet, except in the blisters (or central regions) of the medicament dose portions and the inner ends of the lid elongate sheet or the blister elongate sheet, as applicable, ideally in such a manner that the lid elongate sheet and the blister elongate sheet can be peeled apart. Optionally, the lid elongate sheet and the blister elongate sheet may be bonded using bonding, welding, or any other method known to a person skilled in the art.

In a variation of the blister strips, two elongate form medicament carriers, operable to dispense two different doses in combination or sequentially to the user, could be combined in a blister strip. Accordingly, the blister strip may comprise a first medicament dose portion and a second medicament dose portion corresponding to the first and second elongate carriers, respectively. The distal end of the first portion has a first end region and the distal end of the second portion has a second end region and there is a length of foil between the two portions. The first end region is proximal to the first medicament dose portion of the first portion. The second end region is proximal to the second medicament dose portion of the second portion. The aforementioned length of foil between the two portions of the blister strip comprises no medicament dose portions. Optionally, the length of foil is at least partially formed into a closed loop. In such arrangement, the length of foil of both the elongate form medicament carriers may be combined together face-to-face to form a unified length and the second end regions of the two elongate form medicament carriers could be combined (or connected) to form a common closed loop. It will be appreciated that the unified length comprises only the length of foil of the blister elongate sheets, while the corresponding segments of the lid elongate sheets are peeled and wound around corresponding bobbins that form part of bobbin units. Moreover, the two elongate form medicament carriers are forked from the unified length at the distal end regions thereof. In such arrangement, the common loop brings the two elongate form medicament carriers into a folded-configuration such that the lid elongate sheet corresponding to one elongate form medicament carrier faces the lid elongate sheet corresponding to the other elongate form medicament carrier. Optionally, the unified length is preferably permanently connected, such as by bonding, welding, and the like, to create such a bond. Beneficially, the unified length enables progression or advancement of the two elongate form medicament carriers manufactured as a single strip.

The term “loop” as used herein refers to a “hair-pin like” structure achievable by folding of the second end region of the distal end of the at least one elongate form medicament carrier. It will be appreciated that the lid elongate sheet may be similar in size to or slightly longer than the blister elongate sheet. Moreover, optionally, the distal end of the lid elongate sheet provides a structure similar to a lift-tab for peeling the lid elongate sheet from the blister elongate sheet to expose the medicament dose portions. Moreover, the distal end of each of the lid elongate sheets is connected to a bobbin, so as to wind the lid elongate sheet onto the bobbin in use. Furthermore, the distal end of the blister elongate sheet, after peeling the lid elongate sheet therefrom, may be harnessed to a dispensing mechanism, such as a take-up spool.

The term “medicament active” as used herein refers to a pharmaceutical compound/drug, a vaccine or genetic material combined with a vector, administered to a user for healing, treating, altering, improving, restoring, relieving, and/or curing a particular condition, disease, or mental or physical state. Notably, the medicament active includes an active ingredient or a combination of active ingredients and inactive ingredients, optionally mixed with an excipient or dissolved in some other carrier for delivery to the user. Furthermore, the mixture of medicament actives comprises a plurality of pharmaceutical compounds, wherein the plurality of pharmaceutical compounds does not react with each other.

Optionally, the medicament active is at least one active pharmaceutical ingredient (API) having a physiological effect when ingested or otherwise introduced into the body of the user (namely, a patient). In an example, the drug is an anti-inflammatory drug (such as a corticosteroid, or any other steroid), or a bronchodilator (such as a Long-acting beta agonist (LABA) or a long-acting muscarinic receptor agonist (LAMA)) for a patient suffering from asthma or chronic obstructive pulmonary disease (COPD). Notably, the bronchodilator as the medicament active is employed for dilating bronchi and bronchioles of the patient it is administered to and for increasing airflow to the lungs. Furthermore, when an anti-inflammatory drug is used as the medicament active component, it is employed to reduce swelling in the patient, decrease airway sensitivity caused by inflammation and reduce mucus production. Optionally, the at least one API may be combined with at least one excipient—an inactive substance that aids in the manufacturing process and/or protects, supports, enhances stability, bioavailability or patient acceptability of an active substance, such as drug or other active substances. Optionally, the excipients serve as a carrier or medium for a drug or other active medicament to reach its target site in the body of the patient. Optionally, excipients may include, but are not limited to, fillers, preservatives, colouring agents, antioxidants, binders, anticoagulating agents, coating agents, emollients, emulsifying agents.

Moreover, the powdered form of the medicament active is available to the user of the medicament dispenser when the medicament dispenser is in use thereby.

Optionally, each medicament dose portion of the elongate form medicament carrier comprises a single active medicament component. Alternatively, each medicament dose portion of the elongate form medicament carrier comprises a plurality (i.e., more than one) of active medicament components, such as in a plurality of sets of medicament dose portions. Optionally, the dose portions in the two or more elongate form medicament carriers may be similar or different in terms of: a type (namely a drug type, such as a bronchodilator or an anti-inflammatory drug) of medicament active, a form (namely, a powdered form, a tablet or a liquid) of medicament active, an amount (namely, a higher amount or a lower amount) of medicament active, a frequency of administering of medicament active, and so forth. It will be appreciated that the dose portions in the two or more elongate form medicament carriers may be chosen with regard to at least one of: patient's age, symptoms, severity of condition, a prescribed medication, and medication side effects.

Moreover, those skilled in the art will recognize that other embodiments for carrying out or practising the present disclosure are also possible. For example, it may be understood by a person skilled in the art that the use of two elongate form medicament carriers is merely an example, which should not unduly limit the scope of the claims herein. The person skilled in the art will recognize that many variations, alternatives, and modifications of embodiments of the present disclosure are possible, such as for example bonding, in hair-pin like loop turn, an end of the single-strip elongate form medicament carrier not containing the medicament dose portions (namely, the length of foil) upon itself so as to result in an additional region for reliably enabling the used single-strip elongate form medicament carrier to be collected.

Moreover, the at least one elongate form medicament carrier is inserted in the medicament dispenser. The term “medicament dispenser” as used herein refers to a portable medicine storage unit for users, that dispenses any number of medications to the users when required. In an embodiment, the medicament dispenser may be implemented as a dry powder inhaler (DPI). The medicament dispenser has a dispensing mechanism which is adapted to operate, upon each actuation of the medicament dispenser, to dispense a medicament active from a medicament dose portion of the at least one elongate form medicament carrier. It will be appreciated that upon each actuation of the medicament dispenser, at least one medicament active is dispensed from at least one medicament dose portion of the at least one elongate form medicament carrier. For example, two medicament dose portions corresponding to each of the two elongate form medicament carriers (manufactured as a single strip), may dispense medicament actives upon each actuation of the medicament dispenser. Moreover, the dispensing mechanism is operable to dispense said medicament active(s) upon actuation of the medicament dispenser by the user. It will be appreciated that the medicament dispenser is provided with an actuation means thereon, wherein the user uses the actuation means to cause actuation of the medicament dispenser to dispense or release the medicament active from the medicament dose portion of the at least one elongate form medicament carrier. In an embodiment, the actuation means is implemented as a mouthpiece cover or a nosepiece cover of the dispenser, wherein the user uncovers and sucks or inhales via the mouthpiece cover or the nosepiece cover respectively, to trigger the release of the medicament dose. In another embodiment, the actuation means is implemented as a slider, or a button, wherein the user slides the slider or presses the button to trigger the release.

Moreover, the dispensing mechanism comprises: at least one power spring mechanism for winding the lid elongate sheet, wherein the at least one power spring mechanism comprises: at least one spring having an inner end about which the at least one spring unwinds, and at least one shaft at which the inner end of the at least one spring is secured, wherein the power spring mechanism tensions and peels a segment of the lid elongate sheet peeled from a corresponding segment of the blister elongate sheet upon each actuation of the medicament dispenser.

In this regard, the power spring mechanism may comprise a tightly wound spring made from a flat strip of spring material that is cut to length and wound around a shaft and which unwinds into a housing (such as a housing defined by a side wall of a bobbin). Specifically, the inner end of the spring may be bent and attached to the shaft (located inside the housing), such that each turn rests on its inner neighbour, and an opposite, free outer end may be attached to the housing, i.e. the side wall of the bobbin. Moreover, the power spring mechanism is designed to store mechanical energy and works by creating rotational energy in either the shaft or the bobbin, producing usable torque. In the disclosed mechanism, the rotational energy is imparted to the bobbin, while the shaft is immobile. Notably, the torque generated increases as the spring is wound tighter and decreases as it unwinds.

The spring may be a coiled spiral spring, based on a ribbon of a springy material. It will be appreciated that the shaft has a compact structure that occupies a small space inside the housing, and a proportion of the rest of the space inside the housing is occupied by the coiled spiral spring. It will be appreciated that the diameter of the shaft with respect to the diameter of the housing affects the available space inside the housing and therefore the number of available turns in the coiled spiral spring (for example 10-35 turns). Additionally, the number of available turns and the material properties and the dimensions (length, width and thickness) of the coiled spiral spring define the performance thereof. Beneficially, the coiled spring may be configured such that the torque exerted by the power spring mechanism, corresponding to the last medicament dose portion dispensed by the device, is above a threshold torque value. Further, the threshold torque value is preferably sufficient to peel the lid elongate sheet from the blister elongate sheet. Furthermore, the threshold torque value is preferably less than the torque value that would cause the first medicament dose portion dispensed from the device to be prematurely opened.

The dispensing mechanism comprises a bobbin, as outlined above. The bobbin forms part of a bobbin unit, and can rotate with respect to the remainder of the bobbin unit. The bobbin is hollow and cylindrical, and an outer end of the at least one spring can be secured at a side wall of the bobbin, as described above. A shaft is positioned within the bobbin and forms an axis of rotation. The preferred form of bobbin is implemented as a hollow and cylindrical case that may be made of a metal or plastic. The hollow and cylindrical bobbin has a cavity within which the at least one spring may be held by the outer end such that it imparts a torque to the bobbin and rotates the bobbin, while the shaft remains immovable inside the bobbin. Notably, the outer end of the spring is anchored to the bobbin at an inner side wall thereof. Moreover, the shaft is positioned within the hollow and cylindrical bobbin to retain the inner end of the spring therein, about which the spring unwinds. As mentioned above, it will be appreciated that the shaft occupies a small space inside the bobbin, and a proportion of the rest of the space inside the bobbin is occupied by the coiled spiral spring.

In such case, the bobbin may anchor the distal end of the lid elongate sheet of the elongate form medicament carrier thereon at or near an outer side wall of the bobbin. Moreover, the bobbin may be configured to wind the lid elongate sheet thereon, starting with the distal end of the lid elongate sheet, and continuing to wind the lid elongate sheet onto the bobbin as the lid elongate sheet is peeled away from the corresponding blister elongate sheet as doses are dispensed from the medicament dose portions. The bobbin is generally barrel-shaped. Preferably, the barrel shape provides a larger diameter centrally than at the edges, corresponding to the centre and edges widthways across the lid elongate sheet. For example, the central diameter may be in the range from 0.1 to 2 mm greater than the edge diameters, e.g. 0.25 to 2 mm, which are preferably equal to each other. The barrel shape improves reliability of consistent winding, avoiding lateral displacement of the lid elongate sheet as it is wound. Herein, the at least one bobbin is capable of a rotational motion to aid winding of the lid elongate sheet thereon.

The bobbin has a rotational axis (formed by the shaft) and the spring is arranged to unwind about the rotational axis of the bobbin. As mentioned before, the shaft is positioned at least partially within the bobbin and oriented parallel to the rotational axis of the bobbin, while the at least one shaft is immobile (cannot rotate) such that the at least one spring imparts rotational energy to the bobbin. Preferably, the spring unwinds about the rotational axis of the bobbin. Unwinding of the spring in a unidirectional manner results in a unidirectional rotation of the bobbin.

The shaft is coaxial with the bobbin. Coaxial arrangement of the shaft inside the bobbin eliminates potential wobbling of the bobbin and makes the medicament dispenser steady during operation. Furthermore, the coaxial arrangement of the shaft inside the bobbin results in a higher number of available turns in the power spring.

As noted above, the shaft is mounted in the medicament dispenser so as to prevent movement of the shaft when the medicament dispenser is in use. In this regard, the spring is retained by at least one of: a ratchet, a clip, a clutch or a stopper. Specifically, the ratchet, the clip, the clutch or the stopper, when arranged in the medicament dispenser, prevent the shaft from rotating as the spring subsequently winds or unwinds during the use of the medicament dispenser upon actuation thereof. Moreover, the power spring mechanism may comprise the ratchet, the clip, the clutch or the stopper, arranged such that the shaft may be locked into place relative to the bobbin during assembly of the power spring mechanism, so that the spring is not driven in either direction during use of the medicament dispenser device.

In this regard, the term “ratchet” as used herein refers to a mechanical device that allows continuous linear or rotary motion in one direction while preventing motion in the opposite direction. Moreover, the ratchet is used as a means of holding the shaft in a tensioned state. Alternatively, the spring and the elongate form medicament carrier may be inserted in a pretensioned state for smooth running of the medicament dispenser. Notably, a ratchet comprises a pawl. The pawl consists of a pivoting bar whose free end engages with the teeth of a cogwheel or gear, so that the cogwheel or gear can only turn or move in one way (namely, direction). It typically engages the gear at a steep angle so that it can be made to ride over the teeth when the cogwheel is turned in the correct direction of rotation, but digs into the teeth when the cogwheel is turned in the incorrect direction of rotation.

The term “clip” as used herein refers to a device that grips, clasps, or hooks an object. The clip is a metal or plastic clip working with the at least one shaft. Specifically, the at least one shaft is pushed into the clip for secured retention thereof, and pulled out to be released. The term “clutch” as used herein refers to a coupling or a locking mechanism used to connect and disconnect a driving and a driven part of a mechanism, such as the dispensing mechanism. The clutch may be a dog clutch (also known as dog gears). The dog clutch is a type of clutch that couples at least shafts or other rotatable components not by friction but by interference or clearance fit. Moreover, the two parts of the clutch are designed such that one will push the other, causing both to rotate at the same speed without slipping. The term “stopper” as used herein refers to a component that brings to a halt or stop an operating or functioning element of the device. The stopper may be used to control or stop the rotation of the at least one shaft, upon each actuation of the medicament dispenser, to dispense a medicament active from the medicament dose.

In use, the spring acts to rotate the bobbin so as to peel a segment of the lid elongate sheet from a corresponding segment of the blister elongate sheet. The term “unwinding” as used herein refers to uncoiling or rotation of the spring to wind about the inner end thereof inside the hollow cylindrical bobbin, thereby peeling the lid elongate sheet with each dose indexing. It will be appreciated that unwinding of the provides rotational energy, via the outer end of the spring, to the bobbin, and, as described above, the spring is attached at an inner side of the side wall of the bobbin. Moreover, at the outer side of the side wall of the bobbin the distal end of the lid elongate sheet, separated from the distal end of the blister elongate sheet, may be attached. Such attachment may be to the outer side wall of the bobbin or to the outer end of the at least one spring. In preferred embodiments the lid (or base) foil is attached to the bobbin component that forms part of a bobbin housing that houses the spring, rather than directly to the spring itself. Preferably, the distal end of the elongate lid sheet is secured to the bobbin, such that the spring acts on the lid sheet via rotation of the bobbin rather than by direct attachment of the lid sheet to the spring element itself. The rotation of the bobbin, in response to the unwinding of the spring, tensions the lid elongate sheet, thus peeling a segment of the lid elongate sheet from a corresponding segment of the blister elongate sheet of the elongate form medicament carrier, the lid elongate sheet and the blister elongate sheet, further separating these upon each actuation of the medicament dispenser. Moreover, for each pre-defined unwinding a corresponding medicament dose portion, sealed by said segment of the lid elongate sheet, is exposed for dispensing the medicament active therein. Notably, the spring undergoes the pre-defined unwinding until a last medicament dose portion is exposed to dispense a last medicament active stored therein.

Optionally, the spring is ribbon-shaped or coiled-ribbon-shaped, such as to take the form of a spiral spring when assembled in the power spring mechanism. The spring winds into a concentrically aligned coil during the assembly of the device and is held by a maximum traction force. The traction force refers to force used to generate motion between a body and a tangential surface by dry friction. During the dispensing of the dose, the spring loses some of its tension due to the reduction of the traction force and unwinds causing the bobbin to rotate and tension the lid elongate sheet thereon. Notably, the power spring mechanism may have any spring, but not limited to, clock spring, coil spring, mainspring, hair spring, wind spring, spiral spring, recoil spring, retractor spring, constant force spring.

Optionally, the spring is fabricated from any one or more of: a metal, an alloy of metal, a plastic, a paper or a rubber. The spring may be fabricated from a metal e.g., copper, iron, beryllium, titanium, and the like. The metal alloys include for example stainless steel, carbon steel, chrome silicon (chromium and silicon alloy), chrome vanadium (chromium and vanadium alloy), elgiloy (cobalt, chromium and nickel alloy) phosphor bronze, brass, and the like. It will be appreciated that the spring could be fabricated from paper, rubber and plastic, for example plastic composites, polyphenylene sulfide, acrylonitrile butadiene styrene (ABS), nylon, acrylic, polyamide-imide (PAI) and the like. Notably, the spring fabrication material desirably has a high strength and a high elastic limit. The springs are designed to undergo large deflections over their usage; the fabrication material must also have an extensive elastic range to perform efficiently throughout use. In an example, the power spring mechanism includes a spring produced from a strip of a flat tempered steel that is wound tightly into a spiral configuration. Typically, the strength of the spring fabrication material, such as for example steel, may be 400 Megapascal (MPa), and the elastic limit of the aforesaid steel-based spring fabrication material, may be 21×10⁴ MPa.

Optionally, the dispensing mechanism further comprises: at least one receiving portion for receiving the at least one elongate form medicament carrier having multiple distinct medicament dose portions, the receiving portion comprising: at least one receiving capstan for receiving the medicament dose portions of the at least one elongate form medicament carrier; and a manifold, associated with at least one receiving capstan, for dispensing the medicament active from each of the medicament dose portions of the at least one elongate form medicament carrier; and a take-up spool for holding a distal end of the blister elongate sheet.

In this regard, the at least one receiving portion generally refers to at least one chamber of the medicament dispenser in which the at least one elongate form medicament carrier is inserted. Specifically, the receiving portion comprises the at least one receiving capstan to receive the medicament dose portions of the at least one elongate form medicament carrier. Optionally, the receiving portion may be implemented as a single receiving portion that receives at least one elongate form medicament carrier, such as two or more elongate form medicament carriers.

Alternatively, optionally, the receiving portion may be implemented as plural distinct receiving portions for receiving respective elongate form medicament carriers. In such case, each of the plural distinct receiving portions comprises a receiving capstan to receive a respective elongate form medicament carrier. In an example, a first receiving capstan in a first receiving portion receives a first elongate form medicament carrier, a second receiving capstan in a second receiving portion receives a second elongate form medicament carrier, and so forth.

Moreover, the receiving capstan is in the form of a dimpled cylinder. More specifically, dimples of the receiving capstan may appear as recesses that register (or position) the medicament dose portions adjacent to the manifold. In this regard, the receiving capstan comprises a plurality of recesses for accommodating multiple distinct medicament dose portions (such that one medicament dose portion is registered in one recess) of the elongate form medicament carrier and a given capstan positions a given medicament dose portion adjacent to the manifold for delivery (such as in a ‘First In First Out’ (FIFO) sequence) thereof. Moreover, upon rotation of the receiving capstan in order to advance a medicament dose portion thereon adjacent to the manifold, the medicament active in the medicament dose portion is dispensed and presented to the manifold.

The manifold is positioned to be in communication in turn with the distinct medicament dose portions releasable by the dispensing mechanism to enable dispensing the medicament active therein to the user. Optionally, the manifold has an opening for inhalation of the dispensed medicament active by the user. Optionally, the manifold may comprise an opening corresponding to the elongate form medicament carrier. In this regard, the term “opening” as used herein refers to a hole or an empty space in the manifold of the medicament dispenser. Typically, the openings are provided adjacent to the receiving capstan that positions a medicament dose portion adjacent to the manifold for delivery thereof. Optionally, a single common manifold passage is provided, which communicates in turn with the distinct medicament dose portions of the elongate form medicament carrier via the respective opening of the manifold. Notably, a single distinct medicament active from the medicament dose portion, adjacent to the manifold, is received into the single common manifold passage for inhalation by the user. Optionally, said common manifold passage is shaped to encourage mixing of said released medicament dose portions. Herein, the term “inhalation” refers to an act of inhaling or drawing air into the lungs for breathing or respiration process of the user. Advantageously, the elongate form medicament carrier is practically airtight around the opening thereof, in order to control airflow to the medicament dose portion to dispense it. In an example, the user may apply suction to the exposed medicament dose portions (once the lid elongate sheet and the blister elongate sheet are separated as a result of the peeling action by the dispensing mechanism) through the opening in the manifold. Optionally, the manifold provides an access (implemented as a mouthpiece or nosepiece of the medicament dispenser) between a compartment of the medicament dispenser housing the elongate form medicament carrier and a chamber for mixing a dispensed dose with an airstream for delivery to a patient. Specifically, when the user applies a suction to a mouthpiece or a nosepiece of the medicament dispenser, due to the change in pressure an airstream (namely, an airflow) is introduced into the medicament dispenser. Such airstream may carry the medicament active from the medicament dose portions via the manifold to the user. Alternatively, optionally, there are two manifolds, one for each of the two elongate form medicament carriers.

The take-up spool winds up the distal end of the blister elongate sheet of the elongate form medicament carrier. It will be appreciated that while inserting the elongate form medicament carrier in the medicament dispenser, the lid elongate sheet and the blister elongate sheet of the elongate form medicament carrier are partially separated at the distal ends thereof and that each of the distal ends is harnessed on a respective bobbin and take-up spool. Specifically, the distal end of the blister elongate sheet is affixed to the take-up spool to spool the used blister elongate sheet thereon, starting with the distal end of the blister elongate sheet and continuing to wind the used portions (i.e. after dispensing doses from the medicament portions) of the blister elongate sheet, upon rotation of the take-up spool. The mechanism of the take-up spool is triggered by the actuation means triggering the release of the medicament active from the respective medicament dose portions. Specifically, the actuation means may trigger the rotation of the take-up spool such that the used blister elongate sheet is wound up around the take-up spool. Optionally, the rotation motion of the take-up spool enables the peeling apart of the lid elongate sheet from the blister elongate sheet. Specifically, a spooling mechanism of the take-up spool results in the used blister elongate sheet coiling in on itself as further portions of the blister elongate sheet are fed through the receiving capstans towards the take-up spool. Optionally, the radius of the take-up spool is less than the radius of the capstan. Notably, a small radius of the take-up spool reduces the length of the elongate form medicament carrier wound thereon as well as the slack condition in the elongate form medicament carrier, thus overcoming a jamming condition.

Optionally, the take-up spool comprises a peg for securing the distal end of the blister elongate sheet. Specifically, the peg is implemented as a short pin or bolt, around which the distal end of the blister elongate sheet is secured. Moreover, the distal end of the blister elongate sheet has a loop which is attached to the peg to spool the used blister elongate sheet. Optionally, the peg is positioned away from the axis of rotation of the take-up spool and rotates by exerting a pressure on the loop of the blister elongate sheet to enable winding of the blister elongate sheet on the take-up spool. The peg is typically positioned parallel to an axis of rotation of the take-up spool. The axis of rotation is the centreline of the take-up spool about which the take-up spool rotates. The rotation of the take-up spool results in a rotational motion of the attached loop of the distal end of the blister elongate form sheet. Notably, the direction of winding of the blister elongate sheet on the take-up spool is opposite to the direction of unwinding of the elongate form medicament carrier from the receiving capstan.

Optionally, the dispensing mechanism is configured to drive the receiving capstan and the take-up spool with gears. The term “gears” as used herein refers to an arrangement, e.g., of intermeshed cog wheels, that is configured to ensure that the medicament dose portion has been exposed upon exit of a specific length of elongate form medicament carrier from the receiving portion. As the arrangement of gears changes a relative position thereof, the corresponding components of the medicament dispenser rotate including the take-up spool, thereby, winding the blister elongate sheet on the take-up spool.

In an exemplary implementation, the gearing arrangement comprises a plurality of gears driven through a cover driver gear. The term “cover driver gear” as used herein refers to a gear that is driven by the opening and or closing of the mouthpiece cover or the nosepiece cover. The cover driver gear is coupled with a plurality of receiving capstan gears, or a single capstan gear. The plurality of receiving capstan gears may comprise a first receiving capstan gear coupled to a second receiving capstan gear, wherein the first receiving capstan gear drives the first receiving capstan and the second receiving capstan gear drives the second receiving capstan. Alternatively, the single capstan gear may drive two or more receiving capstans, such as the first receiving capstan and the second receiving capstan. Furthermore, the cover driver gear is coupled to a take-up spool gear wherein, the take-up spool gear drives the take-up spool using a connecting gear.

Optionally, the power spring mechanism peels the segment of the lid elongate sheet corresponding to the receiving capstan receiving a medicament dose portion. It will be appreciated that the gear arrangement of the medicament dispenser enables the bobbin, the receiving capstan and the take-up spool to rotate. Notably, rotation of the bobbin, achieved by the power spring mechanism, results in the spring undergoing the pre-defined unwinding, thereby peeling a segment of the lid elongate sheet corresponding to the segment of the elongate medicament sheet between a pair of adjacent medicament dose portions. It will be appreciated that the medicament dose portions are held sequentially in the recesses of the receiving capstan, and the friction of engagement between the receiving capstan and the gear arrangement prevents the spring from rapidly unwinding and peeling the lid elongate sheet too far from the blister elongate sheet. Therefore, high tension in the spring is made possible, and the lid elongate sheet is only peeled by an amount presented by indexing of the medicament dose portions, even when such high tension is applied. Moreover, it will be appreciated that the size or diameter of the bobbin remains the same size throughout the usage of the medicament dispenser while the outer diameter of the spooled lid elongate sheet increases as the used lid elongate sheet is collected. Therefore, the bobbin is required to have a reducing angular rotation to ensure consistent lid elongate sheet advancement lengths as the spooled lid elongate sheet diameter grows, for sequential indexing of the medicament dose portions throughout the usage of the medicament dispenser. The disclosed mechanism provides for such reducing angular rotation automatically.

Optionally, the medicament dispenser has walls that surround the elongate form medicament carrier and also provide a peel point for directing the lid elongate sheet away from the trajectory provided for the blister sheet around the receiving capstan, such as the first or the second receiving capstan. The relative positions of the peel point and the receiving capstan, the tension provided by the power spring mechanism and the adhesion of the lid elongate sheet to the blister elongate sheet result in the angle at which the lid elongate sheet is peeled from the blister elongate sheet. Such an angle may be measured between the tensioned lid elongate sheet, between the peel point and the receiving capstan, and a tangent to the blister sheet, nearest to where the lid elongate sheet is peeled, at the receiving capstan. Preferably, said angle is in a range from 70° to 110°. The said angle may for example be from 70, 80, 90 or 100° up to 80, 90, 100 or 110°.

Moreover, the tension in the lid elongate sheet can be adjusted to ensure that the blister elongate sheet is pulled into the dimples of the receiving capstan closer to be presented to the manifold to dispense the medicament actives from the blisters, thereby impacting the location of the blister, and that there is a relatively airtight engagement between the blister elongate sheet and the manifold. Optionally, besides the tension in the lid elongate sheet, the geometry around the peel point and the receiving capstan also contributes to the airtight engagement between the blister elongate sheet and the manifold. Therefore, various components of the medicament dispenser, such as the tension in the lid elongate sheet, the manifold, the receiving capstan and the peel point, impact the exact location of the blisters with respect to the manifold. It will be appreciated that the torque/force required to drive the lid elongate sheet to be peeled and held taut by the power spring mechanism is mostly provided by the tension in the lid elongate sheet (and the take-up spool that holds taut the corresponding part of the used blister elongate sheet), with the role of the capstan being largely to position the blisters for presenting the medicament active therein.

Optionally, the shaft is arranged on an axis parallel to the axis of rotation of the receiving capstan. In this regard, the shaft may be coaxial with the bobbin. Preferably, the respective sides of the bobbin and the receiving capstan, through which the respective axes pass orthogonally, are in planar alignment with each other. It will be appreciated that the shaft and the receiving capstan may be arranged near to each other.

Optionally, the medicament dispenser comprises an indexer that individually indexes the medicament dose portions of the elongate form medicament carrier for dispensing the medicament active. Ideally, the indexer is configured on the medicament dispenser to provide a count of the number of doses remaining. Herein, the indexer is coupled to the aforementioned dispensing mechanism, wherein the indexer indexes a change in count of administered medicament dose portions as the medicament actives are released therefrom by the dispensing mechanism. The indexer enables the user to (a) keep a count of the number of doses remaining, and (b) determine whether the medicament actives have been effectively released from the elongate form medicament carrier and are ready to be taken up, as the number on the indexer changes as soon as the medicament actives have been released by the dispensing mechanism to show the doses remaining.

Notably, the indexer may comprise an arrangement of gears driven by a predefined extent of travel per medicament dose portion. In a simplified implementation of the medicament dispenser, the indexer employs a mechanical counter, typically comprising a series of disks mounted on an axle, with the digits zero to nine marked on edges of the disks, to keep count of the medicament dose portions released from the elongate form medicament carrier. Optionally, the doses remaining are counted in ones, fives or tens, for example. Optionally, the doses remaining are counted in fives. Beneficially, counting in fives makes it easier for printing the numbers on the mechanical counter counting mechanisms. Alternatively, optionally, the indexer is implemented as an electronic counter.

Optionally, a single indexer indexes for the medicament dose portions of one or more elongate form medicament carriers, as the medicament dose portions on the one or more elongate form medicament carriers are always released and administered to the patient simultaneously, therefore the number of administered medicament actives from the one or more elongate form medicament carriers will always be the same.

Alternatively, optionally, plural distinct indexers index the medicament dose portions of one or more elongate form medicament carriers. Specifically, plural (namely, a pair of) distinct indexers are employed in an instance when the medicament dose portions of the first elongate form medicament carrier and the second elongate form medicament carrier are released sequentially. Therefore, since the count of medicament dose portions released from the first and second elongate form medicament carriers are different, distinct indexers are employed to index the corresponding medicament dose portions. Optionally, said plural distinct indexers are mutually coupled. Notably, the plural distinct indexers are coupled such that the count shown by the indexers is in tandem with the medicament dose portions released from the medicament dose portions corresponding to the first and second elongate form medicament carriers.

Alternatively, plural (namely, a pair of) distinct indexers are employed in an instance when the medicament dose portions of the first elongate form medicament carrier and the second elongate form medicament carrier are released simultaneously.

Optionally, the medicament dispenser comprises grip elements configured to flatten the elongate form medicament carrier after dispensing the medicament actives from the medicament dose portions thereof. Specifically, the grip elements operate in a manner similar to a mangle, wherein the grip elements squeeze the medicament dose portions in the used blister elongate sheet together. Alternatively, the grip elements act against a plate and thus squeeze the blister elongate sheet through either side of the plate.

Optionally, any or all components of the dispensing mechanism are drivable by an electronic drive system. Specifically, the medicament dispenser may comprise an electronic drive system housed therein coupled with a power source (such as a battery), wherein the electronic drive system may drive the components such as that of the dispensing mechanism, and optionally, may further drive the rotation of the components such as the receiving capstan and/or the take-up spool.

Optionally, the medicament dispenser additionally comprises an electronic data management system. Optionally, such electronic data management system comprises control instructions for the electronic drive system. Furthermore, optionally, the electronic data management system may store a count of the administered medicament dose portions, details of the medicament actives in the first and second set of medicament dose portions, and timings when the medicament dose portions are to be administered and have been administered.

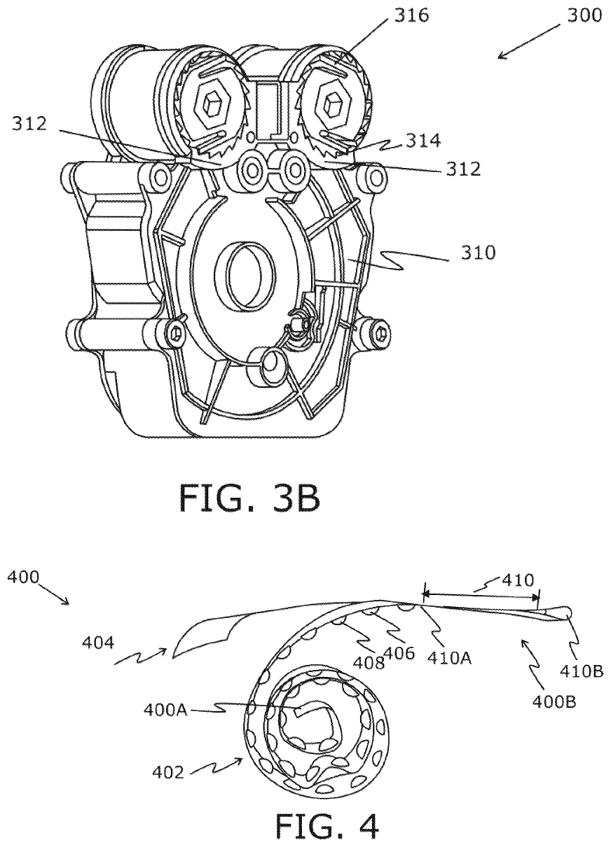

Optionally, the medicament dispenser comprises a body; a holder, shaped to fit within said body and movable relative to said body; and receivable by said holder, a reload cassette containing one elongate form medicament carrier. Specifically, the reload cassette is insertable into the holder (as shown in FIG. 3) after removal of the existing cassette carrying the existing elongate form medicament carrier. Optionally, the medicament dispenser is in kit of parts form. Specifically, the kit comprises the body, the holder and the reload cassette, wherein the user may load the reload cassette into the holder to engage the elongate form medicament carrier with the medicament dispenser and may start the administration of the medicament dose portions thereafter.

In an example, the elongate form medicament carrier is implemented as a peelable blister strip form medicament carrier and the dispensing mechanism comprises a peeling action for peeling apart the lid elongate sheet from the blister elongate sheet. In an instance when the dispensing mechanism provides the peeling action, the distal end of the lid elongate sheet (that is harnessed on a bobbin) and the distal end of the blister elongate sheet (that is harnessed on a take up spool) progressively separate the further portions of the lid elongate sheet and the blister elongate sheet in diverging paths. The medicament active may then be delivered in an airstream, which is typically provided when the user inhales through the medicament dispenser's nosepiece or mouthpiece. Alternatively, the dispensing mechanism may further comprise a blade arranged between the separated portions of the lid elongate sheet and the blister elongate sheet to progressively separate the further portions thereof.

The present disclosure also relates to the method as described above. Various embodiments and variants disclosed above apply mutatis mutandis to the method.

Optionally, an elongate form medicament carrier may be inserted into a medicament dispenser of the disclosure during manufacture. Optionally, the elongate form medicament carrier is assembled into a cartridge (or cassette). The cartridge is designed to insert into a body of the medicament dispenser. The cartridge may comprise a feed spool, a receiving capstan, a take-up spool, and a bobbin. In an exemplary process, a first elongate form medicament carrier is wound onto a first feed spool and a second elongate form medicament carrier is wound onto a second feed spool. A distal end of the lid elongate sheet of the first elongate form medicament carrier is lifted separate from the distal end of the corresponding blister elongate sheet and attached to the circumference of a first bobbin, and similarly, a distal end of the lid elongate sheet of the second elongate form medicament carrier is lifted separate from the distal end of the corresponding blister elongate sheet and attached to the circumference of the second bobbin. Such a process may be carried out on a carriage with spindles that allow the feed spools and bobbins to be positioned for transfer to a unit of the medicament dispenser. The distal ends of the blister elongate sheets of the first and the second elongate form medicament carriers are dragged and harnessed to the take-up spool, specifically, looping the distal end onto a peg of the take-up spool, and arranging the other parts of the first and second elongate form medicament carriers to be fed onto first and second receiving capstans, respectively.

It will be appreciated that assembling the power spring mechanism of the medicament dispenser requires inserting a spring into a bobbin such that an inner end of the spring is secured to the shaft, wherein the shaft is at least partially positioned within the bobbin for securing the inner end of the spring, and an outer end of the at least one spring is secured at a side wall of the bobbin. The inner end of the spring may be secured directly or indirectly to the shaft. The outer end of the at least one spring may be secured to a side wall of the bobbin. The at least one bobbin (with the spring within it) is inserted into the medicament dispenser. Moreover, the inner end of the spring (secured to its position by the at least one shaft and the outer end of the at least one spring affixed on the inner side wall of the at least one bobbin) enables the pre-defined tension within the spring to be maintained.

Furthermore, the distal end of the lid elongate sheet is harnessed on the outer surface of the bobbin, and similarly the distal end of the blister elongate sheet is harnessed on the take-up spool. Moreover, the bobbin is rotated to coil a segment (namely, a pre-defined length, or a length within a pre-determined range) of the lid elongate sheet around the bobbin, until the lid elongate sheet is pulled taut. It will be appreciated that said coiling of the segment of the lid elongate sheet around the bobbin prevents the anchor point of the lid elongate sheet (on the outer surface of the bobbin) from becoming detached by the high force required to peel the lid elongate sheet from the blister elongate sheet during the use of the medicament dispenser. Additionally, said coiling of the segment of the lid elongate sheet around the bobbin ensures that there is no slack in the lid elongate sheet upon first use of the medicament dispenser. Furthermore, during the actuation of the medicament dispenser by the user, the spring unwinds thereby rotating the bobbin which peels a segment of the lid elongate sheet simultaneously from each of the elongate form medicament carrier and exposes the medicament active stored in the medicament dose portions of the elongate form medicament carrier for access into the manifold from when a user inhales through the mouthpiece or nosepiece. In such instance, the bobbin and the take-up spool spools the separated lid elongate sheet and the blister elongate sheet, respectively.

In an implementation of the present disclosure, the medicament dispenser comprises the take-up spool and the pair of bobbins employed to wind up the blister elongate sheet and lid elongate sheet, respectively. In such implementation, the first receiving capstan and the second receiving capstan are driven by a mechanism which coordinates their rotation with the rotation of the take-up spool. Specifically, the mechanism coordinating the rotation ensures the coordinated rotation of the take-up spool and the pair of receiving capstans such that for every actuation of the medicament dispenser, a single distinct medicament dose portion from each of the first and second sets of medicament dose portions is dispensed by peeling apart the lid elongate sheet and the blister elongate sheet.

Optionally, the spring is pre-tensioned for insertion thereof in the bobbin. It will be appreciated that the spring is pre-tensioned during assembly, and then unwinds, thereby reducing the tension in the spring, throughout the use of the medicament dispenser. Moreover, as the spring unwinds the lid elongate sheet is pulled in a direction divergent from the direction of pull of the blister elongate sheet, causing the lid elongate sheet to be peeled from the blister elongate sheet. In such instance, the medicament dose portions sealed between the lid elongate sheet and the blister elongate sheet are exposed to dispense the medicament actives therein, as the medicament dose portions are indexed into position by the user.

Optionally, a unidirectional spring winding rotation of the shaft is retained by at least one of: a ratchet, a clip, a clutch or a stopper.

### DETAILED DESCRIPTION OF THE DRAWINGS

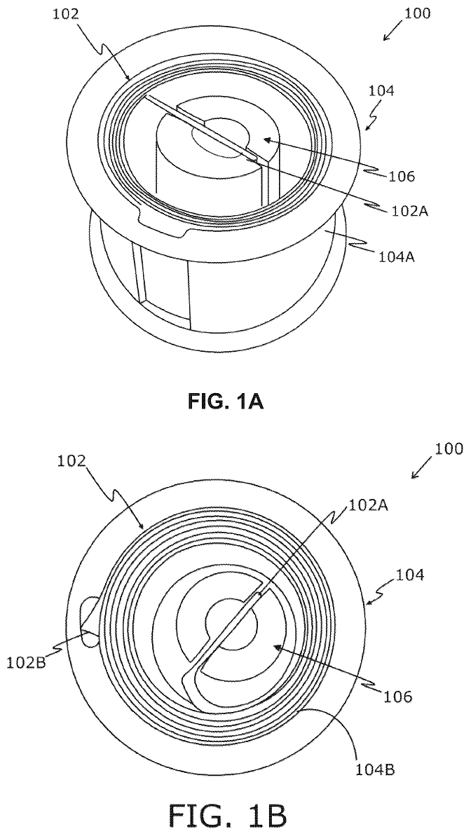

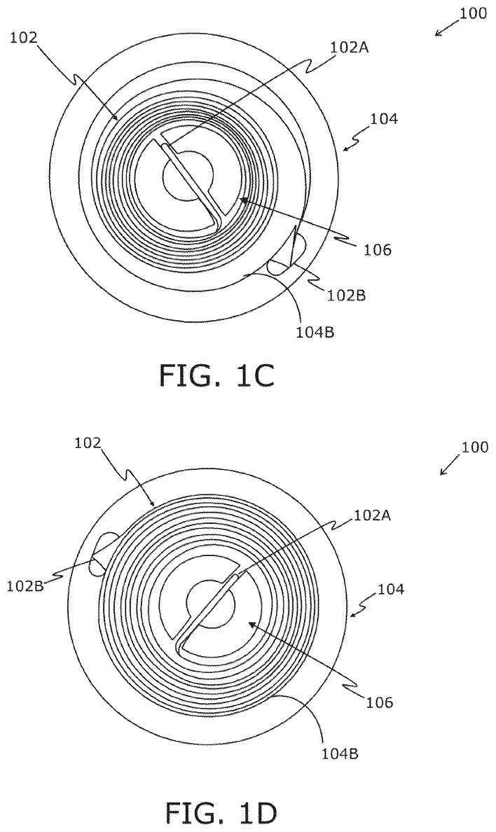

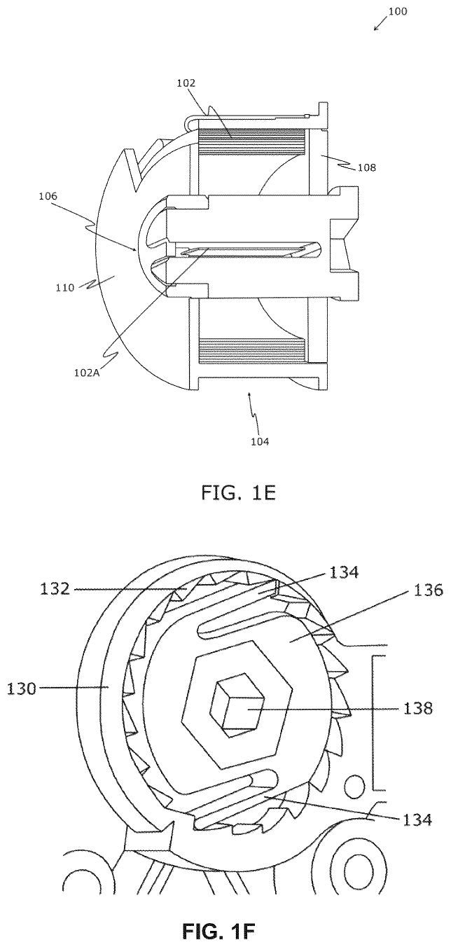

Referring to FIGS. 1A, 1B, 1C and 1D, illustrated are schematic views of a power spring mechanism 100, in accordance with an embodiment of the present disclosure. The power spring mechanism 100 comprises a spring 102 having an inner end 102A about which the spring 102 unwinds, and a bobbin 104 which is hollow and cylindrical, an outer end 102B of the spring 102 being secured at a side wall 104A of the bobbin 104. As shown, the spring 102 is ribbon-shaped or coiled-ribbon-shaped, such as to take the form of a spiral spring when assembled in the power spring mechanism 100. Moreover, the spring 102 is pre-tensioned for insertion thereof in the bobbin 104. Furthermore, the spring 102 is arranged to unwind about the rotational axis of the bobbin 104. In the embodiments of the invention described and shown in this specification, the power spring 102 is initially charged to a higher energy state during manufacture or assembly. During use, each actuation and dose advancement allows the power spring 102 to unwind by a defined amount, thereby providing energy to rotate the bobbin (e.g. bobbin 1016, 2016, 3016, etc) and apply peeling tension to the lid elongate sheet (e. g sheet 1005, 2005, 3005, etc). As a result, the stored energy and available torque of the power spring 102 may reduce progressively over the service life of the device as the spring 102 moves from the charged state toward a less stressed state.

The embodiments of power spring assembly 100 are configured so that, over the intended number of doses, the remaining spring torque remains sufficient to generate the peeling tension required to separate the lid elongate sheet (e. g sheet 1005, 2005, 3005, etc) and wind the separated lid elongate sheet.

By contrast, in some prior art dispensers, the take up spring is tensioned during each dose cycle by an indexing drive or transmission such that the spring is repeatedly wound to restore its stored energy. In such arrangements, the spring primarily acts as an accumulator whose stored energy is replenished during discrete dose advancement events.

Moreover, the power spring mechanism 100 comprises a shaft 106 at least partially positioned within the bobbin 104 for securing the inner end 102A of the spring 102. As shown, the shaft 106 is coaxial with the bobbin 104. In this specification, references to a “shaft” include any axle, spindle, pin, trunnion, journal, boss, or other rotational support about which the bobbin or hub rotates. The shaft may be a separate component fixed relative to the housing or may be formed integrally with the housing. In other embodiments the shaft is integrally formed with the bobbin or hub, for example as one or more axial projections, and the bobbin or hub is received in corresponding bearing features, apertures or recesses in the housing to provide rotational support.

FIG. 1A illustrates a perspective view of the power spring mechanism 100. As shown, the spring 102 as supplied is assembled into the bobbin 104, wherein the inner end 102A of the spring 102 is secured directly or indirectly to the shaft 106. The coils of the spring 102 are in their most expanded state within the bobbin 104, as this is the assembly position before tensioning.

The power spring mechanism may be regarded as a combined take up and peeling actuator. The assembly converts stored energy in the power spring into a peeling torque at the bobbin and a tensile force in the lid elongate sheet. During dose advancement, the blister strip may be advanced by an indexing mechanism, however the work of peeling the lid elongate sheet from the blister elongate sheet is provided by the power spring mechanism via spring biased bobbin rotation. In this manner, the indexing mechanism is not required to provide a separate peeling drive for separating the lid elongate sheet from the blister elongate sheet.

In some known blister-based dispensers a spring biased take up spool is used primarily to remove slack or accommodate an increasing winding diameter as spent material is collected. In contrast, in embodiments described herein the power spring driven bobbin is configured to apply a controlled peeling tension in the lid elongate sheet that is sufficient to perform the peeling of the lid elongate sheet from the blister elongate sheet, while simultaneously winding the separated lid elongate sheet onto the bobbin. Thus, the bobbin assembly performs both the separation of the lid elongate sheet and its subsequent collection.

FIG. 1B illustrates a top view of the power spring mechanism 100, i.e. the same state of tension as in FIG. 1A. As shown, the spring 102 rests against an inside face 104B of the bobbin 104. It will be appreciated that the spring 102 is already in a tensioned state in the bobbin 104. Notably, the spring 102 has tension but is stable and unable to release its stored energy, because there is no space for the coils of the spring 102 to expand into.

FIG. 1C illustrates a top view of power spring mechanism 100, after almost fully tensioning. The spring 102 tensioning corresponds to the medicament dose portion registered with a manifold of a medicament dispenser being dose portion number ‘zero’, i.e., before the first dose is actuated. As shown, the spring 102 is mostly tightly coiled around the shaft 106.

FIG. 1D illustrates a top view of the power spring mechanism 100. As shown, the spring 102 has a residual tension when the medicament dose portion registered with the manifold is dose portion number ‘thirty’, i.e., the last medicament dose portion has been registered with the manifold. As shown, the spring 102 coils are not fully returned to their rest position, i.e., against the inside face 104B of the bobbin 104, thus indicating a residual tension in the power spring mechanism 100. By using part of the available tension in the spring 102 to dispense all doses (before the spring reaches the condition shown in FIGS. 1A and 1B), the torque applied through life does not tail off towards the last dose.

Referring to FIG. 1E, illustrated is a sectional schematic view of the power spring mechanism 100, in accordance with an embodiment of the present disclosure. As shown, the shaft 106 secures the inner end 102A of the spring 102. Moreover, the shaft 106 is mounted in the medicament dispenser in a manner to prevent movement of the shaft 106 when the medicament dispenser is in use. As shown, the shaft 106 is retained for ease of rotation by retaining means 108, 110. As shown, the retaining means 108 and 110 are walls of the holder 300 as explained in FIGS. 3A and 3B below.

The elongate sheet 112 is shown at a stage before it is peeled apart where it has sealed blisters 114. After peeling to provide a base elongate sheet 116 and a lid elongate sheet 118, the base elongate sheet has open blisters 120.

The shaft 106 is shown with a hexagonal end, and a similar hexagonal end is provided on the rear side of the power spring mechanism. The power spring mechanism is inserted into a medicament dispenser, which may be provided with a ratchet mechanism, such as shown in FIG. 1F, that engages the hexagonal end of the shaft, allowing it to rotate in only one direction relative to the medicament dispenser.

FIG. 1F is a view of the ratchet mechanism from the opposite side with respect to the power spring mechanism, and hence designed to rotate anticlockwise. A ring extension 130 of the medicament dispenser has teeth 132 arranged circumferentially on an inner surface of the ring. Each tooth has a long surface and a short surface. A ratchet pawl carrier 136 is provided with a mounting comprising a hexagonal orifice 138. The ratchet pawl carrier 136 has a pair of pawls 134, that are directed at the short surfaces of two diametrically opposite teeth. When the pawl carrier is rotated in the allowed direction, the pawls ride over the long surfaces of the teeth.

In one method of assembly, the power spring mechanism is attached to the distal end of an elongate sheet. It is then mounted into the medicament dispenser such that a hexagonal end of the shaft is engaged in the hexagonal orifice. Other non-circular cooperating shapes for the end of the shaft and the orifice are possible, e.g., rectangles, cross shapes. The spring is then tensioned by turning the other end of the hexagonal shaft, until a prescribed torque is reached. Once tensioned, no further adjustments of the shaft are made in use of the medicament dispenser.

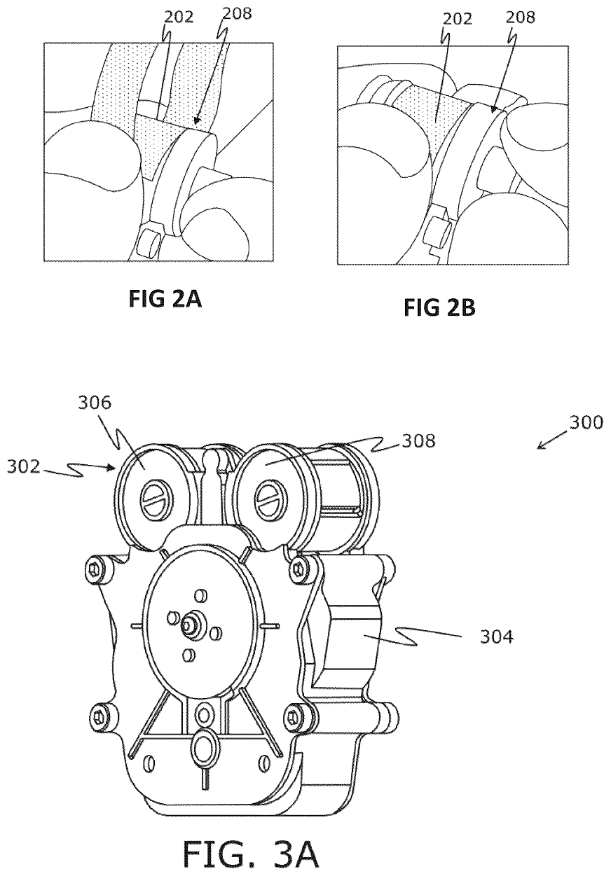

Referring to FIGS. 2A and 2B, illustrated are schematic illustrations of one possible example of the steps 200 for anchoring and tensioning a distal end 202A of the lid elongate sheet 202 by or near to a circumference 208A of the bobbin 208, in accordance with an embodiment of the present disclosure. These FIGs. show hand assembly, and show the fingers and thumb of the person performing the assembly operation. It will be appreciated that at this stage the distal end of the blister elongate sheet (not shown) is harnessed on a take-up spool (not shown). Moreover, as shown in FIGS. 2A and 2B, the bobbin 208 is rotated to coil a segment (namely, a pre-defined length) of the lid elongate sheet 202 around the bobbin 208, until the lid elongate sheet 202 is pulled taut. Furthermore, during subsequent actuation of the medicament dispenser by a user, the spring 204 unwinds thereby rotating the bobbin 208 to peel further segments of the lid elongate sheet 202 from corresponding segments of the blister elongate sheet simultaneously to dispense medicament active stored in a medicament dose portions of the elongate form medicament carrier (not shown).

Referring to FIGS. 3A and 3B, illustrated are external views of opposite sides of a holder 300 of a medicament dispenser, in accordance with an embodiment of the present disclosure. The holder 300 holds the at least one elongate form medicament carrier. The holder 300 is housed within an outer housing (not shown) of the medicament dispenser. The holder 300 has a casing part 302, which has walls 304 that surround the elongate form medicament carrier and a pair of drum ends 306, 308 for a pair of power spring mechanisms, in a unitary moulding. The drum ends 306, 308 provide mountings for a second end of each shaft, such as the shaft 106 as explained in FIG. 1, of the pair of power spring mechanisms. The holder 300 has a lid part 310, which encloses the elongate form medicament carrier by abutting the edges of the walls of the casing part 302. The lid part 310 is moulded to include a pair of rings 312 of inwardly-directed ratchet teeth 314 for engaging corresponding pawls 316 that are attached to corresponding shafts.

Referring to FIG. 4, illustrated is a perspective view of an elongate form medicament carrier 400, in accordance with an embodiment of the present disclosure. The elongate form medicament carrier 400 has a proximal end 400A, a distal end 400B, and a blister elongate sheet 402 and a lid elongate sheet 404 enclosing multiple distinct medicament dose portions, depicted as the exemplary medicament dose portions 406, and 408, therebetween. As shown, the multiple distinct medicament dose portions are uniformly spaced. Notably, the distal end 400B of the elongate form medicament carrier 400 does not comprise the multiple distinct medicament dose portions, and appears as a flat length of foil 410 having a first end region 410A and a second end region 410B. Optionally, the second end region 410B is a looped end.

It may be understood by a person skilled in the art that FIG. 4 illustrates one simplified implementations of the elongate form medicament carrier 400, for the sake of clarity, which should not unduly limit the scope of the claims herein. The person skilled in the art will recognize many variations, alternatives, and modifications of embodiments of the present disclosure.

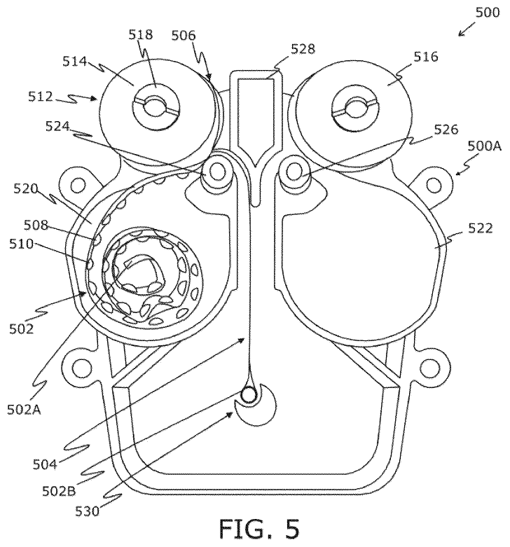

Referring to FIG. 5, illustrated is a sectional view of part of a medicament dispenser 500, in accordance with an embodiment of the present disclosure. Shown is the inner side of the first part 500A of the medicament dispenser 500. The medicament dispenser 500 holds an elongate form medicament carrier 502, similar to the elongate form medicament carrier 400 explained in FIG. 4. The elongate form medicament carrier 502 has a proximal end 502A, a distal end 502B, and a blister elongate sheet 504 and a lid elongate sheet 506 enclosing multiple distinct medicament dose portions, depicted as the exemplary medicament dose portions 508, and 510, therebetween. Moreover, the medicament dispenser 500 has a dispensing mechanism which is adapted to operate, upon each actuation of the medicament dispenser 500, to dispense a medicament active from a medicament dose portion of the elongate form medicament carrier 502. The dispensing mechanism comprises a power spring mechanism 512 (such as the power spring mechanism 100 explained in FIG. 1) for winding the lid elongate sheet 506. Moreover, the power spring mechanism comprises a spring (not shown) having an inner end about which the spring unwinds, and an outer end secured at a side wall of a bobbin, depicted as (514 and 516), wherein the bobbin 514 (and 516) is a hollow cylindrical bobbin. Furthermore, the power spring mechanism 512 comprises a shaft 518 at least partially positioned within the bobbin 514 (and 516) for securing the inner end of the spring. Moreover, a pre-defined unwinding of the spring spools a segment of the lid elongate sheet 506 peeled from a corresponding segment of the blister elongate sheet 504, to be tensioned by the bobbin 514 (and 516).

The dispensing mechanism further comprises at least one receiving portion, depicted as the receiving portion 520 and 522, for receiving the at least one elongate form medicament carrier, such as the elongate form medicament carrier 502, having multiple distinct medicament dose portions. Moreover, the receiving portion comprises at least one receiving capstan, depicted as the receiving capstan 524 and 526, for receiving the medicament dose portions of the at least one elongate form medicament carrier; and a manifold 528, associated with at least one receiving capstan, for dispensing the medicament active from the medicament dose portion of the at least one elongate form medicament carrier. The dispensing mechanism further comprises a take-up spool 530 for holding a distal end 502B of the blister elongate sheet 504.

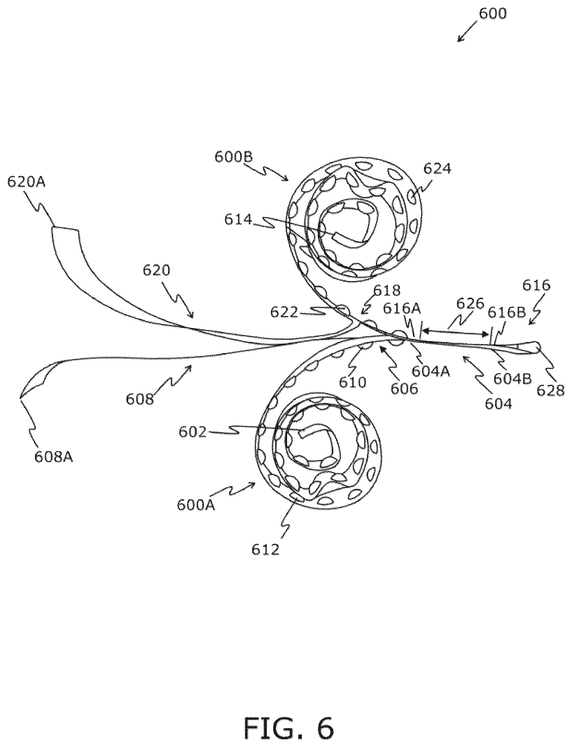

Referring to FIG. 6, illustrated is a perspective view of an elongate form medicament carrier 600, in accordance with an embodiment of the present disclosure. The elongate form medicament carrier 600 is implemented as an elongate strip form medicament carrier with a two-portion elongate form medicament carrier comprising a first portion 600A and a second portion 600B. The first portion 600A has a proximal end 602, a distal end 604, and a blister elongate sheet 606 and a lid elongate sheet 608 enclosing multiple distinct medicament dose portions, depicted as the exemplary medicament dose portions 610 and 612, therebetween. Similarly, the second portion 600B has a proximal end 614, a distal end 616, and a blister elongate sheet 618 and a lid elongate sheet 620 enclosing multiple distinct medicament dose portions, depicted as the exemplary medicament dose portions 622 and 624, therebetween. As shown, the multiple distinct medicament dose portions are uniformly spaced. Moreover, the each of the distal ends 604 and 616 of the first portion 600A and the second portion 600B, respectively, do not comprise the multiple distinct medicament dose portions, and appear as a flat length of foil having a first end region 604A and 616A and a second end region 604B and 616B, respectively. Furthermore, the length of foils of both the first portion 600A and the second portion 600B are combined together face-to-face to form a unified length 626 and the second end regions 604B and 616B thereof are combined (or connected) to form a common loop 628. Moreover, the unified length 626 comprises only the length of foil of the blister elongate sheets 606 and 618 of the first portion 600A and the second portion 600B, respectively, while the corresponding segments of the lid elongate sheets 608 and 620 of the first portion 600A and the second portion 600B, respectively, are peeled therefrom and harnessed to the corresponding bobbins (not shown). Furthermore, the first portion 600A and the second portion 600B appear to be forked at the respective first end regions 604A and 616A thereof. In such arrangement, the common loop 628 brings the two-portion elongate form medicament carrier into a folded configuration such that the lid elongate sheet 608 corresponding to the first portion 600A faces the lid elongate sheet 620 corresponding to the second portion 600B. Moreover, the lid elongate sheets 608 and 620 have distal ends 608A and 620A, respectively, wherein the distal ends 608A and 620A are not secured to the blister elongate sheets 606 and 618 and are later harnessed to the corresponding bobbins.

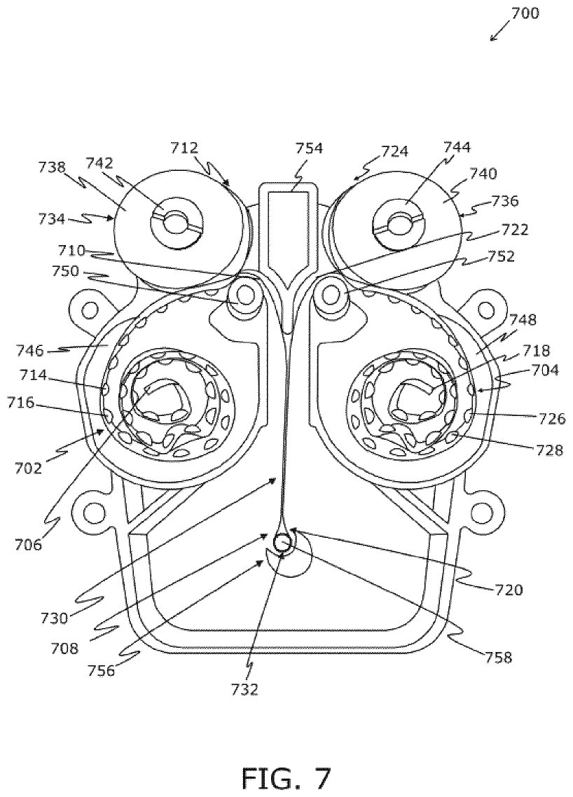

Referring to FIGS. 7 and 8, illustrated are sectional views of part of a medicament dispenser 700, in accordance with another embodiment of the present disclosure. The medicament dispenser 700 holds at least one elongate form medicament carrier (such as the elongate form medicament carrier explained in FIG. 6), comprising a first portion 702 and a second portion 704 (such as the first portion 600A and a second portion 600B explained in FIG. 6). The first portion 702 has a proximal end 706, a distal end 708, and a blister elongate sheet 710 and a lid elongate sheet 712 enclosing multiple distinct medicament dose portions, depicted as the exemplary medicament dose portions 714 and 716, therebetween. Similarly, the second portion 704 has a proximal end 718, a distal end 720, and a blister elongate sheet 722 and a lid elongate sheet 724 enclosing multiple distinct medicament dose portions, depicted as the exemplary medicament dose portions 726 and 728, therebetween. Moreover, a certain length of the blister elongate sheets 710 and 722 are combined together face-to-face to form a unified length 730 wherein the corresponding lid elongate sheets 712 and 724 are peeled apart from said length of the blister elongate sheets 710 and 722. The unified length 730 ends in a loop 732 formed by combining (or connecting) distal ends 708 and 720.

Moreover, the medicament dispenser 700 has a dispensing mechanism which is adapted to operate, upon each actuation of the medicament dispenser 700, to dispense a medicament active from a medicament dose portion of the at least one elongate form medicament carrier. The dispensing mechanism comprises at least one power spring mechanism 734 and 736 (such as the power spring mechanism 100 explained in FIGS. 1A-1E) for winding the lid elongate sheet 712 and 724. Moreover, the at least one power spring mechanism 734 and 736 comprises at least one spring (such as the spring 102 explained in FIGS. 1A-1E) having an inner end about which the at least one spring unwinds, and an outer end secured at a side wall of a bobbin, such as the bobbins 738 and 740, wherein the bobbin is a hollow cylindrical bobbin, and at least one shaft, such as the shafts 742 and 744, at least partially positioned within the bobbin 738 and 740, respectively, for securing the inner end of the at least one spring, wherein a pre-defined unwinding of the at least one spring spools a segment of the lid elongate sheet 712 and 724 peeled from a corresponding segment of the blister elongate sheet 710 and 722, to be tensioned by the bobbin, such as the bobbin 738 and 740.

The dispensing mechanism further comprises at least one receiving portion, such as the receiving portions 746 and 748, for receiving the at least one elongate form medicament carrier 702 and 704 respectively. The receiving portion 746 and 748 comprises at least one receiving capstan, such as the receiving capstans 750 and 752, for receiving the medicament dose portions of the at least one elongate form medicament carrier 702 and 704 respectively; and a manifold 754, associated with at least one receiving capstan 750 and 752, for dispensing the medicament active from the medicament dose portion of the at least one elongate form medicament carrier 702 and 704 respectively. The dispensing mechanism further comprises a take-up spool 756 for holding distal ends 708 and 720 of the blister elongate sheet 710 and 722, that together form the loop 732. Moreover, the take-up spool 756 comprises a peg 758 that secures the loop 732.

As shown in FIG. 8, as the take-up spool 756 is rotated, starting with the loop 732 (see FIG. 7), the take-up spool 756 continues to wind the used portions of the blister elongate sheet 710 and 722 thereon after dispensing medicament actives from the medicament dose portions. During the said process, as the receiving capstans 750 and 752 are rotated, starting with the distal ends (not shown) of the lid elongate sheet 712 and 724 the pair of bobbins 738 and 740 continue to wind thereon the portions of the lid elongate sheet 712 and 724 corresponding to the used portions of the blister elongate sheet 710 and 722 after dispensing medicament active from the medicament dose portions.

Modifications to embodiments of the present disclosure described in the foregoing are possible without departing from the scope of the present disclosure as defined by the accompanying claims. Expressions such as “including”, “comprising”, “incorporating”, “have” and “is” used to describe and claim the present disclosure are intended to be construed in a non-exclusive manner, namely allowing for items, components or elements not explicitly described also to be present. Reference to the singular is also to be construed to relate to the plural.

### Attachment Variations

The following additional attachment methods may be used to affix the distal end of the lid foil or elongate sheet to the bobbin, or a bobbin-associated feature, or similar.

### Heat Sealing

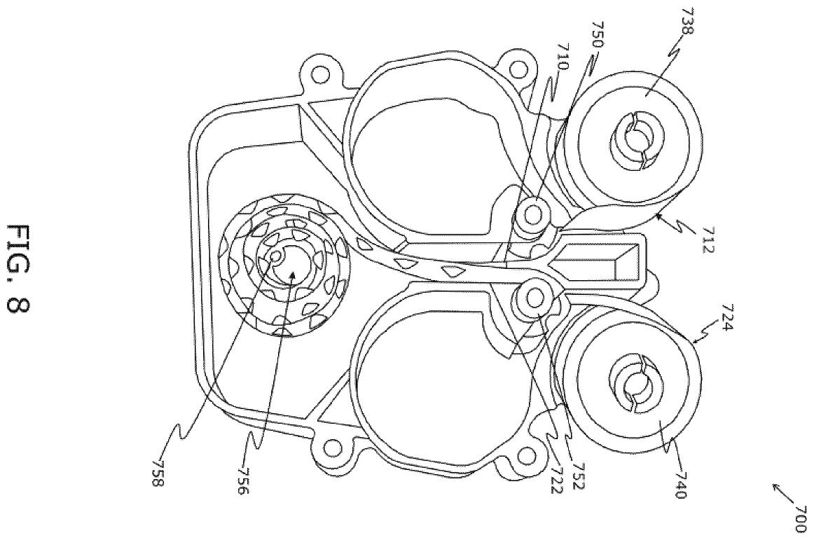

FIG. 9 shows two elongate form medicament carriers 1002, and bobbin units 1006, 1008 that form part of a medicament dispenser.

Each medicament carrier 1002 has the form of an elongate ribbon, and comprises a blister elongate sheet 1004 and a lid elongate sheet 1005 that enclose multiple distinct medicament dose portions therebetween. The medicament carriers 1002 have a proximal end 1002A and a distal end (as shown in FIG. 9, the blister elongate sheet 1004 and a lid elongate sheet 1005 have been partly peeled or separated from one another. The distal end for an unpeeled or complete medicament carrier would be formed by the distal end 1005B of the lid elongate sheet 1005 and the distal end 1004B of the blister elongate sheet 1004).

Use will now be described with reference to a single one of the carriers 1002, and an associated single one of the bobbin units—unit 1008. Operation of the second carrier and bobbin unit 1006 is substantially the same.

In use, upon each actuation of the medicament dispenser, the elongate form medicament carrier 1002 associated with bobbin unit 1008 is peeled apart so as to dispense a dose portion from the blisters in the medicament carrier 1002, and the lid elongate sheet 1005 is wound around a bobbin 1016 that forms part of the bobbin unit 1008, being ‘taken up’ around the bobbin 1016.

In this embodiment or variation, a heat sealed connection is made between the outer surface of the bobbin 1016 and the distal end 1005B of the lid elongate sheet 1005. The outer end 1005B of the lid elongate sheet 1005 is heat-sealed to the bobbin 1016. The bobbin 1016 in this variation incorporates a flat or gently contoured thermal sealing land 1020 (a specific area of the surface where the seal is formed). The land 1020 is configured to receive the distal end 1005B of the lid elongate sheet 1005. The lid elongate sheet 1005 in this variation has a laminate structure that comprises a heat-activatable polymer layer which is configured to soften under the application of controlled temperature above room temperature. The lid elongate sheet 1005 is placed in contact with the surface of the bobbin 1016 at the land 1020, and is then heat-activated (for example by using a rotary heated tool to apply circumferential heat for rapid sealing). This causes the elongate sheet 1005 to bond to the surface of the bobbin 1016. Heat may be applied through the lid elongate sheet or directly to the bobbin face.

The surface of the bobbin 1016 may optionally include micro-texturing to improve the bond with the lid elongate sheet.

Attachment in this manner has the benefit of providing a clean, particulate-free joint suitable for drug-handling environments, with no added materials required.

It should be noted that although attachment in this manner has been described for a medicament dispenser containing two carriers, this can also be carried out on other forms of dispenser such as those that only contain a single carrier.

### Taping

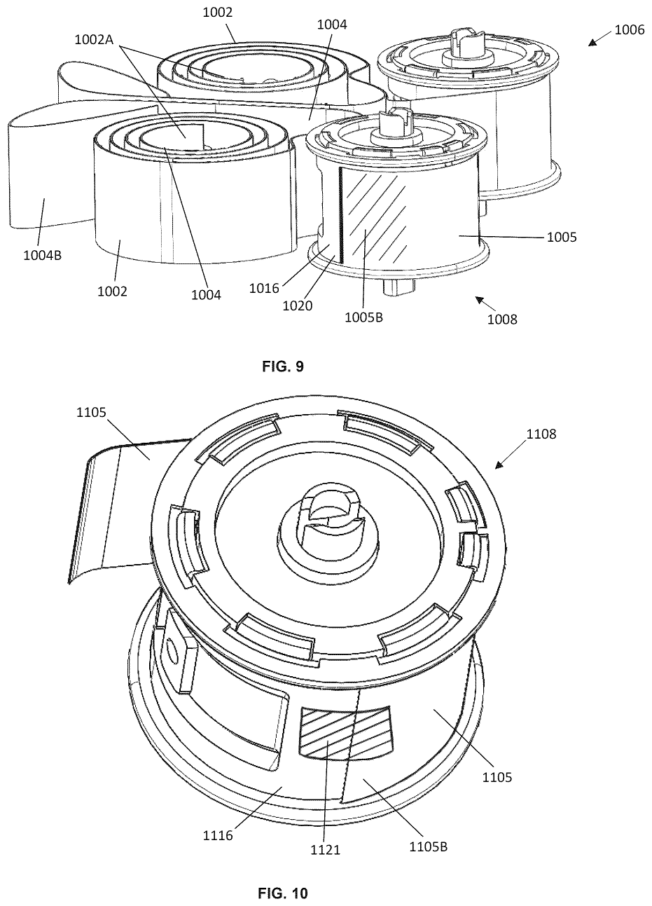

FIG. 10 shows a further variation or additional attachment method. A bobbin unit 1108 and lid elongate sheet 1105 are shown in FIG. 10. These are in general form and in-use operation substantially similar to those described above with reference to FIG. 9.

In this variation, the lid elongate sheet 1105 is wound around a bobbin 1116 that forms part of the bobbin unit 1108, being ‘taken up’ around the bobbin 1116 upon actuation of the medicament dispenser.

The distal end 1105B of the lid elongate sheet 1105 is taped to the bobbin using medical-grade tape. A section 1121 of medical-grade tape or adhesive film is applied to the bobbin 1116, and the end 1105B of the elongate sheet 1105, providing immediate bonding without heat. A double-sided acrylic tape can be used if required. Silicone-based tapes are preferred (but not required) as these provide low-energy surfaces. It is preferred that pre-laminated tape is used on the lid elongate sheet 1105 during preparation of the foil or elongate sheet.

In the variation shown in FIG. 10, the tape 1121 is applied over the outer surface of lid elongate sheet 1105 at the end 1105B of the elongate sheet 1105, extend from the end onto the surface of the bobbin 1116.

This variation is most useful where low mechanical stress is expected during winding. This variation allows bonding at ambient temperatures, and the tape layer may act as a shock absorber, reducing tear initiation.

### Ultrasonic Welding

FIG. 11 shows a further variation or additional attachment method. In this variation, the elongate form medicament carrier 1202 and bobbin units 1206, 1208 are substantially the same as that described above for the ‘heat sealing’ variation (that shown in FIG. 9), with the medicament carriers 1202 and bobbin units 1206, 1208 forming part of a medicament dispenser, and each of the elongate form medicament carriers 1202 comprising a blister elongate sheet 1204 and a lid elongate sheet 1205 that enclose multiple distinct medicament dose portions therebetween. The medicament carriers 1202 have a proximal end 1202A and a distal end (as shown in FIG. 11, the blister elongate sheet 1204 and a lid elongate sheet 1205 have been partly peeled or separated from one another. The distal end for an unpeeled or complete medicament carrier would be formed by the distal end 1205B of the lid elongate sheet 1205 and the distal end 1204B of the blister elongate sheet 1204).

In use, upon each actuation of the medicament dispenser, the elongate form medicament carrier 1202 associated with bobbin unit 1208 is peeled apart so as to dispense a dose portion from the blisters in the medicament carrier 1202, and the lid elongate sheet 1205 is wound around a bobbin 1216 that forms part of the bobbin unit 1208, being ‘taken up’ around the bobbin 1216.

In this variation, the lid elongate sheet 1205 is welded ultrasonically to the bobbin 1216. The lid elongate sheet 1205 comprises a polymer component, or a mechanical interlock. The end 1205B of the lid elongate sheet 1205 is positioned on the bobbin 1216, and ultrasonic energy is then applied at the interface between the lid elongate sheet 1205 and the bobbin 1216 so as to either melt the surface of the polymer component of the lid elongate sheet 1205, or the mechanical interlock. This causes bonding with the bobbin 1216.

If required, the bobbin 1216 can be formed with energy-directing ribs (not shown). The lid elongate sheet 1205 can if required be pre-clamped to the bobbin 1216 so as to maintain tension during the welding process. The welds may be spot-based or continuous rings.

This method of connection has the benefit of creating a strong, repeatable mechanical bond. The method can be caried out quickly and repeatably and is therefore suitable for high-volume production. No added materials are required.

It should be noted that although attachment in this manner has been described for a medicament dispenser containing two carriers, this can also be carried out on other forms of dispenser such as those that only contain a single carrier.

### Adhesive/chemical Bonding

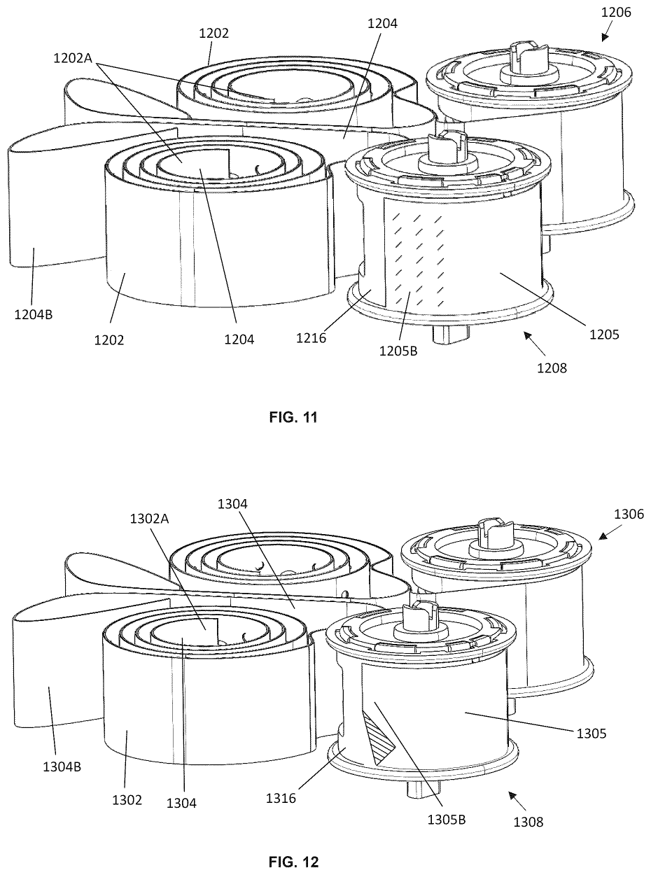

FIG. 12 shows a further variation or additional attachment method. In this variation, the elongate form medicament carrier 1302 and bobbin units 1306, 1308 are substantially the same as that described above for the ‘heat sealing’ variation (that shown in FIG. 9), with the medicament carrier 1302 and bobbin units 1306, 1308 forming part of a medicament dispenser, and each of the elongate form medicament carriers 1302 comprising a blister elongate sheet 1304 and a lid elongate sheet 1305 that enclose multiple distinct medicament dose portions therebetween. The carrier 1302 has a proximal end 1302A and a distal end (as shown in FIG. 12, the blister elongate sheet 1304 and a lid elongate sheet 1305 have been partly peeled or separated from one another. The distal end for an unpeeled or complete medicament carrier would be formed by the distal end 1305B of the lid elongate sheet 1305 and the distal end 1304B of the blister elongate sheet 1304).

In use, upon each actuation of the medicament dispenser, the elongate form medicament carrier 1302 associated with bobbin unit 1308 is peeled apart so as to dispense a dose portion from the blisters in the medicament carrier 1302, and the lid elongate sheet 1305 is wound around a bobbin 1316 that forms part of the bobbin unit 1308, being ‘taken up’ around the bobbin 1316.

In this variation, adhesives are used to connect the lid elongate sheet 1306 and the bobbin 1316. Liquid adhesives such as sprayable adhesives can be used. Alternatively, printable adhesives may be used. The adhesive may be solvent-based or reactive (UV, moisture, or heat cured).

The adhesive can be applied either before or after the lid elongate sheet is positioned on the bobbin. For example, the adhesive can be applied to the underside of the lid elongate sheet 1306 by peeling this backwards as shown in FIG. 12.

If required, micro-dosing nozzles can be used to apply the adhesive so as to generate a consistent bead volume. The adhesive can be applied selectively to create “flex zones” in the lid elongate sheet. Chemical primers may be used as required in order to enhance bond strength.

This method of connection has the benefit of producing a very flexible bond that is compatible with a wide range of bobbin materials. This method is particularly useful for bonding on uneven or contoured bobbin surfaces.

It should be noted that although attachment in this manner has been described for a medicament dispenser containing two carriers, this can also be carried out on other forms of dispenser such as those that only contain a single carrier.

### Mechanical Loop

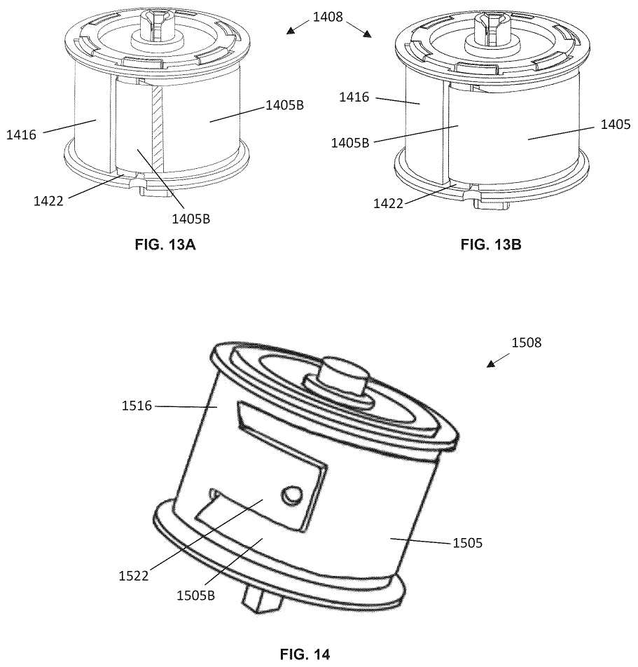

FIGS. 13A and 13B show a further variation or additional attachment method. A bobbin unit 1408 that comprises a bobbin 1416, and a lid elongate sheet 1405, are shown in FIGS. 13A and 13B. These are in general form and in-use operation substantially similar to those described above with reference to FIG. 9—the lid elongate sheet 1405 and bobbin unit 1408 are substantially the same as that described above for the ‘heat sealing’ variation, and operate in the same manner as part of a medicament dispenser, with differences as described below.

In this variation, the lid elongate sheet 1405 and the bobbin 1416 are connected as follows: the bobbin 1416 is formed so as to comprise a feature such as for example a bobbin peg, flange or post 1422 around which the lid elongate sheet 1405 can be passed so as to wrap the end 1405B of the lid elongate sheet 1405 around the bobbin peg, flange or post 1422. The lid elongate sheet is then doubled back on itself and secured to itself using a securing method such as for example heat sealing, adhesive, tape or crimping.

The loop of the lid elongate sheet 1405 may be partial or a full 360 degrees. The peg, flange or post 1422 can if required be formed so as to comprise a low-friction coating. This assists with avoiding scoring of the lid elongate sheet during winding. The lid elongate sheet 1405 can if required include an embossed region to guide folding.

This method of connection has the benefit of transferring loads through a self-reinforcing geometry. That is, the bond does not rely solely on adhesive strength. As well as this, there is increased tolerance to peel forces during use, and reduced reliance on surface chemistry.

### Physical Clipping of the Lid Elongate Sheet to the Bobbin

FIG. 14 shows a further variation or additional attachment method. A bobbin unit 1508 that comprises a bobbin 1516, and a lid elongate sheet 1505, are shown in FIG. 14. These are in general form and in-use operation substantially similar to those described above with reference to FIG. 9—the lid elongate sheet 1505 and bobbin unit 1508 are substantially the same as that described above for the ‘heat sealing’ variation, and operate in the same manner as part of a medicament dispenser, with differences as described below.

In this variation, the bobbin 1516 of the bobbin unit 1508 is formed with an integral mechanical clip 1522. When the lid elongate sheet 1505 is positioned on the bobbin 1516, the clip 1522 on the 1516 directly grips the lid elongate sheet 1505 and attaches this to the bobbin 1516.

In variations, radial clip arms, axial clip collars, snap-fit rings, or hinged features can be used in place of the mechanical clip 1522, either singly or in combination. Alternatively, two-stage locking can be used, where the lid elongate sheet 1505 is first located in the required position and then locked to the bobbin 1516.

This method of connection has the benefit of eliminating the need for adhesives, heat, and curing processes. The method is compatible with high-speed automated insertion. The method further provides a deterministic mechanical retention force.

### Spiked Bobbin

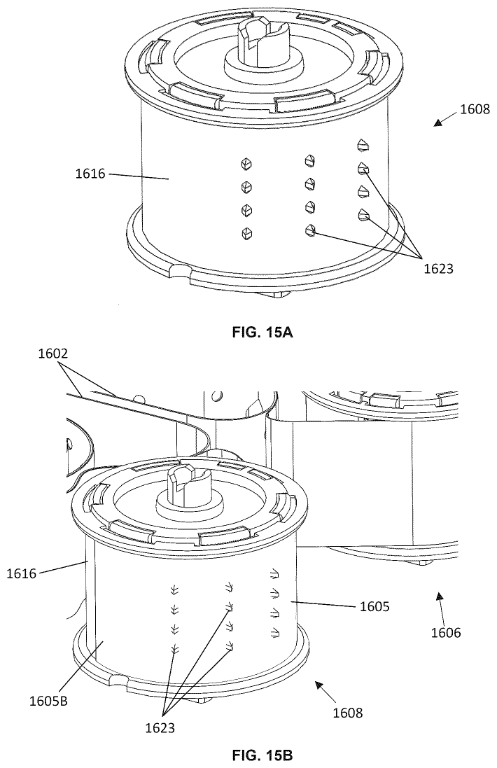

FIGS. 15A and 15B show a further variation or additional attachment method, with FIG. 15A showing a bobbin unit 1608 in isolation, and FIG. 15B showing the bobbin unit 1608 in use with a lid elongate sheet 1605, and other elements of a medicament dispenser substantially similar to those described for the variation above, and in particular the ‘heat sealing’ variation as shown in FIG. 9.

In this variation, the elongate form medicament carrier 1602 and bobbin units 1606, 1608 are substantially the same as that described above for the ‘heat sealing’ variation (that shown in FIG. 9), with the medicament carrier 1602 and bobbin units 1606, 1608 forming part of a medicament dispenser, and each of the elongate form medicament carriers 1602 comprising a blister elongate sheet (not shown) and a lid elongate sheet 1605 that enclose multiple distinct medicament dose portions therebetween.

In use the elongate form medicament carriers are peeled apart so as to dispense a dose portion from the blister elongate sheet. Lid elongate sheet 1605 is peeled away from its associated blister elongate sheet, with the lid elongate sheet 1605 then wound around bobbin 1616 that forms part of the bobbin unit 1608, being ‘taken up’ around the bobbin 1616.

In this variation, the bobbin 1616 is configured to have a plurality of short moulded or machined spikes or barbs 1623 protruding from the surface of the bobbin 1616. When the lid elongate sheet 1605 is located on the bobbin 1616, the spikes or barbs 1623 pierce the lid elongate sheet 1605 and retain it in position on the bobbin 1616.

The use of multiple small spikes 1623 helps to distribute the load. If required, the spikes or barbs 1623 can be angled so as to assist with resisting unwinding forces.

In a variation, blunted micro-spines can be used so as to create friction with the lid elongate sheet 1605 without penetration of the lid elongate sheet 1605.

This method of connection has the benefit of creating a very strong mechanical connection. There is immediate engagement during assembly. No added materials or curing are required.

### Peg Drive Bobbin With Lid Elongate Sheet Apertures

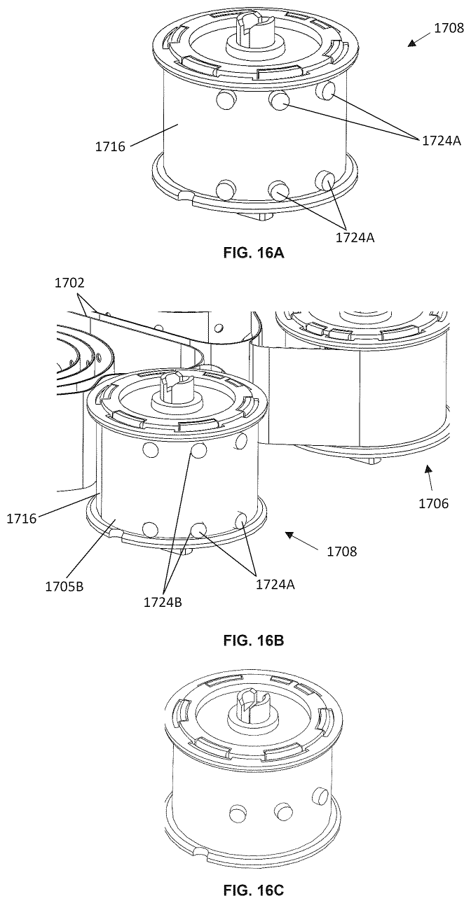

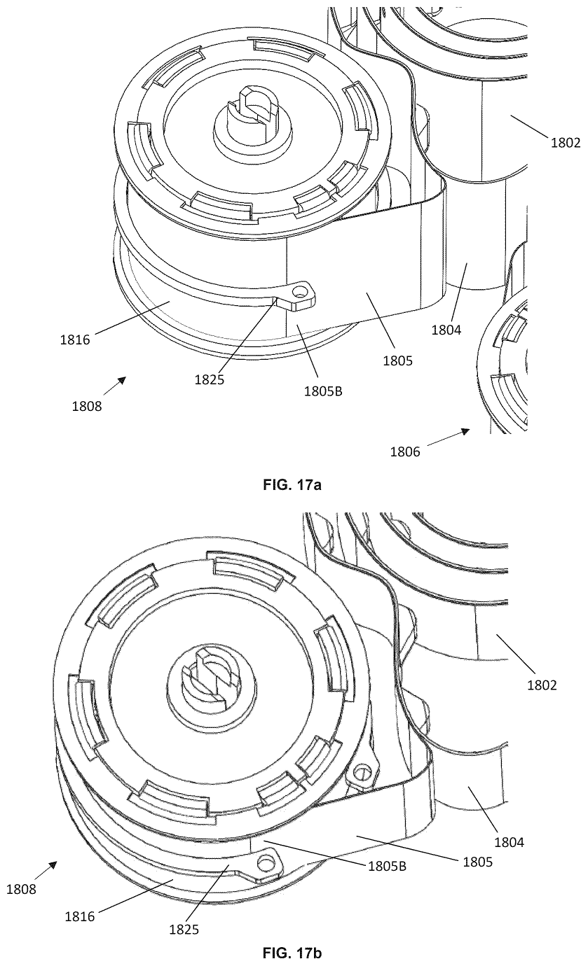

FIGS. 16A and 16B show a further variation or additional attachment method, with FIG. 16A showing a bobbin unit 1708 in isolation, and FIG. 16B showing the bobbin unit 1708 in use with a lid elongate sheet 1705, and other elements of a medicament dispenser substantially similar to those described for the variation above, and in particular the ‘heat sealing’ variation as shown in FIG. 9.

In this variation, the elongate form medicament carrier 1702 and bobbin units 1706, 1708 are substantially the same as that described above for the ‘heat sealing’ variation (that shown in FIG. 9), with the medicament carrier 1702 and bobbin units 1706, 1708 forming part of a medicament dispenser, and each of the elongate form medicament carriers 1702 comprising a blister elongate sheet (not shown) and a lid elongate sheet 1705 that enclose multiple distinct medicament dose portions therebetween.

In use the elongate form medicament carriers are peeled apart so as to dispense a dose portion from the blister elongate sheet. Lid elongate sheet 1705 is peeled away from its associated blister elongate sheet, with the lid elongate sheet 1705 then wound around bobbin 1716 that forms part of the bobbin unit 1708, being ‘taken up’ around the bobbin 1716.

In this variation, the bobbin 1716 is configured so as to comprise pegs 1724A, and the lid elongate sheet 1705 is configured to comprise pre-cut holes or perforations 1724B, complementary to the pegs 1724A. The pegs 1724A engage with the perforations 1724B in the lid elongate sheet 1705 in a similar manner to the mechanism used for sprocket-driven photographic film.

This variation can be implemented with single or multiple arrays of pegs 1724A—for example the variation of peg layout shown in FIG. 16C. Staggered peg spacing can be used for anti-slip control.

It is preferred that the holes or perforations are pre-formed (e.g. by punching into/through the lid elongate sheet 1705 to pre-form the holes/perforations 1724B and prepare the lid elongate sheet 1705). However, the holes/perforations 1724B can also be formed during the assembly process—during bobbin insertion.

This method of connection has the benefit that positive drive (via the pegs 1724A and holes/perforations 1724B) prevents slippage of the lid elongate sheet 1705 during winding.

This type of connection is suitable for high torque or high-spring-force designs. As a further advantage, the hole spacing may be used as a feature for calibration or alignment.

### Circlip Retention

FIG. 17 shows a further variation or additional attachment method. In this variation, the elongate form medicament carrier 1802 and bobbin units 1806, 1808 are substantially the same as that described above for the ‘heat sealing’ variation (that shown in FIG. 9), with the medicament carrier 1802 and bobbin units 1806, 1808 forming part of a medicament dispenser, and each of the elongate form medicament carriers 1802 comprising a blister elongate sheet 1804 and a lid elongate sheet 1805 that enclose multiple distinct medicament dose portions therebetween.

In use, upon each actuation of the medicament dispenser, the elongate form medicament carrier 1802 associated with bobbin unit 1808 is peeled apart so as to dispense a dose portion from the blisters in the medicament carrier 1802, and the lid elongate sheet 1805 is wound around a bobbin 1816 that forms part of the bobbin unit 1808, being ‘taken up’ around the bobbin 1816

In this variation, the bobbin 1016 is formed with a recess (not shown). During assembly, the lid elongate sheet 1805 is placed in position on the bobbin 1816, and a wire circlip or snap ring 1825 is then applied over the lid elongate sheet 1805, pressing it into the recess.

A full circular circlip or partial ring can be used. The recess may include friction-enhancing features. The lid elongate sheet 1805 may be tensioned prior to installation of the circlip 1825.

Circlips are a well-known and well-established reliable type of component, so this method of connection has high reliability. As well as this, this method of connection has the benefit of providing high retention strength with minimal added bulk. The lid elongate sheet is compressed rather than pierced.

### Shaped Lid Elongate Sheet Interlocks With Bobbin

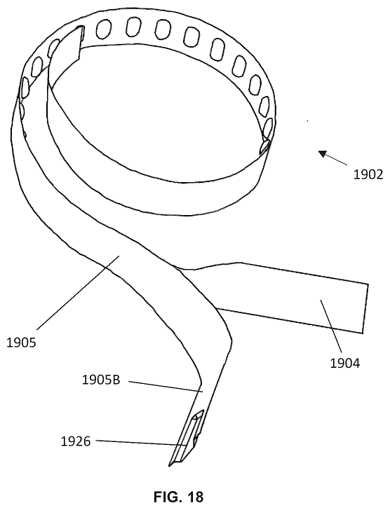

An elongate form medicament carrier 1902 is shown in FIG. 18. The elongate form medicament carrier 1902 comprises a blister elongate sheet 1904 and a lid elongate sheet 1905 that enclose multiple distinct medicament dose portions therebetween.

In this variation, the end 1905B of the lid elongate sheet 1905 comprises a formed recess 1926. The bobbin (not shown) is formed with a geometry complementary to the recess 1926—for example a peg or similar that extends from the surface of the bobbin. During assembly, the lid elongate sheet 1905 is positioned on the bobbin, and the bobbin and lid elongate sheet 1905 interlock with one another via the formed recess 1926 and the complementary geometry on the bobbin.

In variations, the recess 1926 can be formed as a protrusion, emboss, or curl.

In variations, the end of the lid elongate sheet 1905B can be formed with a ‘cup’ shape that extends from the end of the lid elongate sheet 1905B, with a complementary cavity formed on the bobbin so that the ‘cup’ shape locates in use into the bobbin cavity to attach the lid elongate sheet 1905 to the bobbin.

In another variation the lid elongate sheet 1905 can be formed with embossed ribs on that side of the lid elongate sheet 1905 that faces towards the bobbin in use, and the bobbin can be formed with complementary channels, so that in use the ribs locate into the channels to attach the lid elongate sheet 1905 to the bobbin.

In another variation the lid elongate sheet can be formed with lid elongate sheet tabs that are bent into undercuts.

This method of connection has the benefit of providing predictable, repeatable alignment of the bobbin and lid elongate sheet. The load is distributed over a larger area than point-features such as pegs. This method is compatible with automation.

### General

Any of the above connection variations and techniques can be performed with either the laminate side or paper side of the lid elongate sheet facing the bobbin.

For any particular variation, the factor or factors that determine a preferred orientation for any particular combination of lid elongate sheet and bobbin include, but are not limited to:
adhesion compatibility;
ultrasonic response;
friction characteristics;
risk of delamination;
cleanliness constraints;
material preference (e.g., polymer side for heat sealing; paper side for adhesive absorption)

The connection variants described above improve manufacturing flexibility and accommodate a range of material constructions, foil or elongate sheet thicknesses, assembly constraints and device performance requirements. Each of the variants described above has distinct characteristics which are advantageous for particular production environments or inhaler configurations.

1. A medicament dispenser for holding at least one elongate form medicament carrier of the type that comprises a blister elongate sheet and a lid elongate sheet that enclose multiple distinct medicament dose portions therebetween, the medicament dispenser comprising a power spring mechanism which is adapted to operate on each actuation of the medicament dispenser so that a medicament dose portion is dispensed from the elongate form medicament carrier, the power spring mechanism comprising:
a bobbin configured to rotate about a central axis;
a power spring configured to in use unwind so as to drive rotation of the bobbin about the central axis in a winding direction;
the bobbin configured to receive a free/distal end of the lid elongate sheet so that in use the free/distal end of the lid elongate sheet is directly connected to the bobbin, and;
the power spring further configured to operate in use to apply tension sufficient to peel the lid elongate sheet from the blister elongate sheet to expose a medicament dose portion, and to wind the peeled lid elongate sheet around the bobbin.

2. A medicament dispenser as claimed in claim 1 wherein tension is applied via the bobbin.

3. A medicament dispenser as claimed in claim 1 wherein the lid elongate sheet and bobbin are configured so that the lid elongate sheet can be heat sealed to the bobbin.

4. A medicament dispenser as claimed in claim 3 wherein the bobbin comprises a flat or contoured thermal sealing land on the outer surface of the bobbin, configured to receive the distal end of the lid elongate sheet.

5. A medicament dispenser as claimed in claim 3 wherein the outer surface of the bobbin is configured with micro-texturing.

6. A medicament dispenser as claimed in claim 3 wherein the lid elongate sheet comprises a laminate structure comprising at least one heat-activatable polymer layer configured to soften under the application of controlled temperature above room temperature.

7. A medicament dispenser as claimed in claim 1 further comprising a section of medical-grade tape, the tape applied to the bobbin and the distal end of the elongate sheet so as to connect the bobbin and distal end of the elongate sheet.

8. A medicament dispenser as claimed in claim 7 wherein the tape comprises double-sided acrylic tape.

9. A medicament dispenser as claimed in claim 1 wherein the lid elongate sheet and bobbin are configured so that the lid elongate sheet can be ultrasonically welded to the bobbin.

10. A medicament dispenser as claimed in claim 9 wherein the bobbin comprises a flat or contoured land configured to receive the distal end of the lid elongate sheet.

11. A medicament dispenser as claimed in claim 9 wherein the surface of the bobbin is configured with micro-texturing.

12. A medicament dispenser as claimed in claim 9 wherein the surface of the bobbin comprises energy-directing ribs.

13. A medicament dispenser as claimed in claim 1 further comprising adhesive configured to connect the lid elongate sheet and the bobbin.

14. A medicament dispenser as claimed in claim 1 wherein the bobbin is configured with a bobbin peg around which the lid elongate sheet can be wrapped to double back on itself.

15. A medicament dispenser as claimed in claim 14 wherein the bobbin peg comprises a low-friction surface.

16. A medicament dispenser as claimed in claim 1 wherein the bobbin comprises an integral mechanical clip configured to directly grip the lid elongate sheet to attach this to the bobbin.

17. A medicament dispenser as claimed in claim 16 wherein the mechanical clip comprises one or more of: at least one radial clip arm; at least one axial clip collar; at least one snap-fit ring; at least one hinged feature.

18. A medicament dispenser as claimed in claim 1 wherein the bobbin comprises a plurality of short spikes/barbs that protrude from the surface of the bobbin so as to in use pierce the lid elongate sheet and retain the sheet in position on the bobbin.

19. A medicament dispenser as claimed in claim 1 wherein the bobbin comprises a plurality of pegs that extend from the surface of the bobbin, and the lid elongate sheet comprises a plurality of complementary perforations that pass through the sheet, the pegs in use engaging with the perforations.

20. A medicament dispenser as claimed in claim 19 wherein the pegs are arranged in a single array.

21. A medicament dispenser as claimed in claim 19 wherein the pegs are arranged in multiple arrays.

22. A medicament dispenser as claimed in claim 1 further comprising a circlip, the bobbin configured with a recess, the free/distal end of the lid elongate sheet placed in the recess, the circlip in use positioned at least partly over the free end of the lid elongate sheet to hold this in position in the recess.

23. A medicament dispenser as claimed in claim 1 wherein the free/distal end of the lid elongate sheet comprises a formed recess and the bobbin is formed with a complementary peg that extends from the surface of the bobbin, the formed recess and peg interlocking in use.

24. A medicament dispenser as claimed in claim 23 wherein the recess is formed as one of: a protrusion; an emboss; a curl.

25. A medicament dispenser as claimed in claim 1 wherein the bobbin is formed with at least one recess, and the lid elongate sheet is formed with at least one complementary extension feature that protrudes from the surface of the sheet, the extension feature(s) and recess(es) interlocking in use.

26. A medicament dispenser as claimed in claim 25 wherein the extension feature comprises a cup extension, and the bobbin is formed with a complementary recess, the cup and recess interlocking in use.

27. A medicament dispenser as claimed in claim 25 wherein the lid elongate sheet is formed with embossed ribs on that side of the lid elongate sheet that faces towards the bobbin in use, the bobbin formed with complementary recess channels configured to receive the ribs so that in use the ribs locate into the channels to attach the lid elongate sheet to the bobbin.
